(19) United States
(12) Patent Application Publication
(10) Pub. No.: US 2012/0202787 A1
(43) Pub. Date: Aug. 9, 2012

(54) Novel Heteroaryl Imidazoles And Heteroaryl Triazoles As Gamma-Secretase Modulators

(75) Inventors: Christopher William am Ende, Mystic, CT (US); Douglas Scott Johnson, Mystic, CT (US); Martin Youngjin Pettersson, Waterford, CT (US); Chakrapani Subramanyam, South Glastonbury, CT (US); Christopher John O'Donnell, Mystic, CT (US)

(73) Assignee: Pfizer Inc.

(21) Appl. No.: 13/501,326

(22) PCT Filed: Oct. 12, 2010
(86) PCT No.: PCT/IB2010/054616

§ 371 Date: Apr. 11, 2012

Related U.S. Application Data
(60) Provisional application No. 61/253,446, filed on Oct. 20, 2009.
(60) Provisional application No. 61/371,821, filed on Aug. 9, 2010.

(51) Int. Cl. A61K 31/4439; C07D 403/04; A61K 31/497; C07D 401/04; A61P 25/28; C07D 413/14; C07D 417/14; C07D 401/14; C07D 409/14; A61P 25/00; C07D 405/14; C07D 403/14

(52) U.S. Cl. 514/210.18; 546/274.1; 514/341; 544/405; 514/255.05

(57) ABSTRACT

Compounds and pharmaceutically acceptable salts of the compounds are disclosed, wherein the compounds have the structure of Formula I;

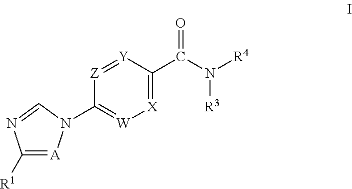

as defined in the specification. Corresponding pharmaceutical compositions, methods of treatment, methods of synthesis, and intermediates are also disclosed.

### FIELD OF THE INVENTION

The present invention relates to the treatment of Alzheimer's disease and other neurodegenerative and/or neurological disorders in mammals, including humans. This invention also relates to the modulation, in mammals, including humans, of the production of A-beta peptides that can contribute to the formation of neurological deposits of amyloid protein. More particularly, this invention relates to heteroaryl imidazole and heteroaryl triazole compounds useful for the treatment of neurodegenerative and/or neurological disorders, such as Alzheimer's disease and Down's Syndrome, related to A-beta peptide production.

### BACKGROUND OF THE INVENTION

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner et al., J. Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Several programs have been advanced by research groups to ameliorate the pathological processes causing dementia, AD, CM and prion-mediated diseases. γ-Secretase modulators are one such strategy and numerous compounds are under evaluation by pharmaceutical groups. The present invention relates to a group of brain penetrable γ-secretase modulators and as such are useful as γ-secretase modulators for the treatment of neurodegenerative and/or neurological disorders related to A-beta peptide production, such as Alzheimer's disease and Down's Syndrome. (see Ann. Rep. Med. Chem. 2007, Olsen et al., 42: 27-47).

### SUMMARY OF THE INVENTION

The present invention is directed to a compound, including the pharmaceutically acceptable salts thereof, having the structure of formula I:

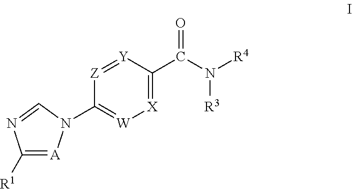

wherein A is CH or N;

W is CR² or N; X, Y, and Z are independently CH or N, and at least one of X, Y, or Z is N;

R¹ is hydrogen, C₁₋₆alkyl, C₃₋₆cycloalkyl, or C₂₋₆alkenyl; wherein said alkyl, cycloalkyl or alkenyl may be optionally substituted with one to three of fluorine, hydroxyl, or C₁₋₆alkoxy groups;

R² is hydrogen, —CF₃, cyano, halogen, C₁₋₆alkyl, or —OR⁵;

R³ and R⁴ are each independently hydrogen, C₁₋₆alkyl, C₂₋₆alkenyl, —(C(R⁶)₂)ₜ-(C₃₋₆cycloalkyl), —(C(R⁶)₂)ₜ-(4- to 10-membered heterocycloalkyl), −(C(R⁶)₂)ₜ-(C₆₋₁₀aryl), or —(C(R⁶)₂)ₜ-(5- to 10-membered heteroaryl); wherein said alkyl, alkenyl, or cycloalkyl, heterocycloalkyl, aryl, or heteroaryl moieties may be optionally independently substituted with one to three R³; or R³ and R⁴ together with the nitrogen to which they are bonded form a 4- to 10-membered heterocycloalkyl optionally substituted with one to three R⁶;

R⁵ is hydrogen, C₁₋₆alkyl, C₃₋₆cycloalkyl, C₃₋₆alkenyl, or C₃₋₆alkynyl; wherein said alkyl, cycloalkyl, alkenyl, or alkynyl may be optionally substituted with cyano, or one to three fluorines;

each R⁶ is independently hydrogen, halogen, cyano, —CF₃, C₂₋₆alkenyl, C₂₋₆alkylidene, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —NHC(O)R⁷, —NR⁷SO₂R⁸, or —N(R⁷)₂; wherein said C₁₋₆alkyl, C₂₋₆alkenyl, C₂₋₆alkylidene, or cycloalkyl, heterocycloalkyl, aryl, or heteroaryl moieties may be optionally independently substituted with one to three R⁹;

each R⁷ is independently hydrogen, C₁₋₆alkyl, —CF₃, —(C(R¹¹)2)ₙ-(C₃₋₆cycloalkyl), —(C(R¹¹)₂)ₙ-(4- to 10-membered heterocycloalkyl), —(C(R¹¹)2)ₙ-(C₆₋₁₀aryl), or —(C(R¹¹)₂)ₙ-(5- to 10-membered heteroaryl); wherein said alkyl, or cycloalkyl, heterocycloalkyl, aryl or heteroaryl moieties may be optionally independently substituted with one to three R¹¹;

each R⁸ is independently C₁₋₆alkyl, —(C(R¹²)₂)ₚ-(C₃₋₆cycloalkyl), —(C(R¹²)₂)ₚ-(4- to 10-membered heterocycloalkyl), —(C(R¹²)₂)ₚ-(C₆₋₁₀aryl), or —(C(R¹²)₂)ₚ-(5- to 10-membered heteroaryl); wherein said alkyl, or cycloalkyl, heterocycloalkyl, aryl, or heteroaryl moieties may be optionally independently substituted with one to three R¹²;

each R⁹ is independently hydrogen, C₁₋₆alkyl, C₂₋₆alkenyl, C₂₋₆alkynyl, halogen, —CF₃, —OR⁷, —(C(R¹⁰)₂)_{q}-(C₆₋₁₀aryl), or —(C(R¹⁰)₂)_{q}-(5- to 10-membered heteroaryl);

each R¹⁰ is independently hydrogen, —CF₃, cyano, halogen, C₁₋₆alkyl, or —OR⁵;

each R¹¹ is independently hydrogen, C₁₋₆alkyl, C₂₋₆alkenyl, C₂₋₆alkynyl, halogen, cyano, —CF₃, or —OCF₃;

each R¹² is independently hydrogen or halogen; and

each t, m, n, p or q is an integer independently selected from 0, 1, 2, 3, and 4.

In another embodiment of the invention, the so-called imidazoles, A is CH.

In another embodiment of the invention, the so-called triazoles, A is N.

In another embodiment of the invention, the so-called (2-amido)-pyridines, X is N, W is CR², and Y and Z are CH; or Y is N, W is CR², and X and Z are CH. Another embodiment of the invention, the so-called (2-amido)-pyridine triazoles, includes the so-called (2-amido)-pyridine embodiment in combination with the “A” triazole embodiment described above. Another embodiment of the invention, the so-called (2-amido)-pyridine imidazoles, includes the so-called (2-amido)-pyridine embodiment in combination with the “A” imidazole embodiment described above.

In another embodiment of the invention, the so-called (5-amido)-pyridines, Z is N, W is CR², and X and Y are CH. Another embodiment of the invention, the so-called (5-amido)-pyridine triazoles, includes the so-called (5-amido)-pyridine embodiment in combination with the “A” triazole embodiment described above. Another embodiment of the invention, the so-called (5-amido)-pyridine imidazoles, includes the so-called (5-amido)-pyridine embodiment in combination with the “A” imidazole embodiment described above.

In another embodiment of the invention, the so-called pyrazines, X and Z are N, W is CR², and Y is CH. Another embodiment of the invention, the so-called pyrazine triazoles, includes the so-called pyrazine embodiment in combination with the “A” triazole embodiment described above. Another embodiment of the invention, the so-called pyrazine imidazoles, includes the so-called pyrazine embodiment in combination with the “A” imidazole embodiment described above.

In another embodiment of the invention, the so called (5-amido)-pyrimidines, W and Z are N, and X and Y are CH. Another embodiment of the invention, the so-called (5-amido)-pyrimidine triazoles, includes the so-called (5-amido)-pyrimidine embodiment in combination with the “A” triazole embodiment described above. Another embodiment of the invention, the so-called (5-amido)-pyrimidine imidazoles, includes the so-called (5-amido)-pyrimidine embodiment in combination with the “A” imidazole embodiment described above.

In another embodiment of the invention, the so-called pyridazines, Y and Z are N, W is CR², and X is CH. Another embodiment of the invention, the so-called pyridazine triazoles, includes the so-called pyridazine embodiment in combination with the “A” triazole embodiment described above. Another embodiment of the invention, the so-called pyridazine imidazoles, includes the so-called pyridazine embodiment in combination with the “A” imidazole embodiment described above.

In another embodiment of the invention, the so called (2-amido)-pyrimidines, X and Y are N, W is CR², and Z is CH. Another embodiment of the invention, the so called (2-amido)-pyrimidine triazoles, includes the so called (2-amido)-pyrimidine embodiment in combination with the “A” triazole embodiment described above. Another embodiment of the invention, the so called (2-amido)-pyrimidine imidazoles, includes the so called (2-amido)-pyrimidine embodiment in combination with the “A” imidazole embodiments described above.

In another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety may be optionally substituted with one to three substituents independently selected from Examples of said heterocycloalkyl include, but are not limited to, azetidine, pyrrolidine, piperidine, and morpholine. In another embodiment, said heterocycloalkyl is substituted with one R³.

In another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety is substituted with one R⁶, and R⁶ is —(C(R⁹)₂)ₘ-(OR⁷), —(C(R⁹)₂)ₘ-(C₆₋₁₀aryl) or —(C(R⁹)₂)ₘ-(5- to 10-membered heteroaryl).

In yet another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety is substituted with one R⁶ and R⁶ is —(C(R⁹)₂)ₘ-(OR⁷) and R⁷ is —(C(R¹¹)₂)ₙ-(C₆₋₁₀aryl); wherein said aryl moiety may be optionally independently substituted with C₁₋₆alkyl, C₂₋₆alkenyl, C₂₋₆alkynyl, halogen, —CF₃, or —OCF₃. Other embodiments of interest to the present inventors are those compounds additionally wherein m is zero.

In yet another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety is substituted with one R⁶ and R⁶ is —(C(R⁹)₂)ₘ-(OR⁷) and R⁷ is —(C(R¹¹)₂)ₙ-(5- to 10-membered heteroaryl); wherein said heteroaryl moiety may be optionally independently substituted with C₁₋₆alkyl, C₂₋₆alkenyl, C₂₋₆alkynyl, halogen, —CF₃, or —OCF₃. Other embodiments of interest to the present inventors are those compounds additionally wherein m is zero.

In another embodiment of the invention R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety is substituted with one R⁶; wherein R⁶ is —(C(R⁹)₂)ₘ-(C₆₋₁₀aryl), and said aryl moiety may be optionally substituted with C₁₋₆alkyl, C₂₋₆alkenyl, C₂₋₆alkynyl, halogen, —CF₃, or —OCF₃. Other embodiments of interest to the present inventors are those compounds additionally wherein m is zero.

In another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, wherein said heterocycloalkyl moiety is substituted with one R⁶; wherein R⁶ is (—C(R⁹)₂)ₘ-(5- to 10-membered heteroaryl), and said heteroaryl may be optionally substituted with C₁₋₆alkyl, C₂₋₆alkenyl, C₂₋₆alkynyl, halogen, —CF₃, or —OCF₃. Other embodiments of interest to the present inventors are those compounds additionally wherein m is zero.

In another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, and said heterocycloalkyl moiety may be optionally substituted with one or two substituents independently selected from R⁶.

In another embodiment of the invention, R³ and R⁴ together with the nitrogen to which they are bonded form a 4- to 10-membered heterocycloalkyl moiety comprising 1 or 2 heteroatoms independently selected from oxygen, nitrogen, or sulfur, and said heterocycloalkyl moiety may be optionally substituted with three substituents independently selected from R⁶.

In any of the embodiments described above, R³ is hydrogen, C₁₋₆alkyl, or —(C(R⁶)2)ₜ-(C₃₋₆cycloalkyl); wherein said alkyl or cycloalkyl moiety may be optionally independently substituted with one to three fluorines; and R⁴ is C₁₋₆alkyl, —(C(R⁶)₂)ₜ-(C₃₋₆cycloalkyl), —(C(R⁶)₂)ₜ-(4- to 10-membered heterocycloalkyl), —(C(R⁶)₂)ₜ-(C₆₋₁₀aryl), or —(C(R⁶)₂)ₜ-(5- to 10-membered heteroaryl); wherein said C₁-₆alkyl, or cycloalkyl, heterocycloalkyl, aryl, or heteroaryl moieties may be optionally independently substituted with one to three substituents independently selected from R⁶. In any of the embodiments described above, R³ is hydrogen, C₁₋₆alkyl, or —(C(R⁶)2)ₜ-(C₃₋₆cycloalkyl); wherein said alkyl or cycloalkyl moiety may be optionally independently substituted with one to three fluorines; and R⁴ is C₁₋₆alkyl wherein said C₁₋₆alkyl R⁴ substituent may be optionally substituted with one to three substituents independently selected from R⁶. In any of the embodiments described above, R³ is hydrogen and R⁴ is C₁₋₆alkyl wherein said C₁₋₆alkyl R⁴ substituent may be optionally independently substituted with one to three R⁶.

In any of the embodiments described above, R³ is hydrogen, C₁₋₆alkyl, or —(C(R⁶)₂)ₜ-(C₃₋₆cycloalkyl); wherein said alkyl or cycloalkyl moiety may be optionally independently substituted with one to three fluorines; and R⁴ is —(C(R⁶)₂)ₜ-(C₃₋₆cycloalkyl); wherein said cycloalkyl moiety may be optionally independently substituted with one to three R⁶. In any of the embodiments described above, R⁴ is —(C(R⁶)₂)ₜ-(C₃₋₆cycloalkyl); wherein said cycloalkyl moiety may be optionally substituted with one R⁶, and R⁶ is selected from C₁₋₆alkyl, —CF₃, fluorine, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —CN, or —N(R⁷)₂. In any of the embodiments described above, R⁴ is —(C(R⁶)₂)ₜ-(C₃₋₆cycloalkyl); wherein said cycloalkyl moiety may be optionally substituted with one R⁶, and R⁶ is fluorine or —CN. In any of the embodiments described above, R⁴ is —(C(R⁶)₂)ₜ-(C₃₋₆cycloalkyl); wherein said cycloalkyl moiety may be optionally substituted with two R⁶ and each R⁶ is independently selected from C₁₋₆alkyl, —CF₃, fluorine, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —CN, or —N(R⁷)₂. In any of the embodiments described above, R⁴ is —(C(R⁶)₂)ₜ-(C₃₋₆cycloalkyl); wherein said cycloalkyl moiety may be optionally substituted with three R⁶, and each R⁶ is independently selected from C₁₋₆alkyl, —CF₃, fluorine, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘm (5- to 10-membered heteroaryl), —(C(R⁹)₂)ₘ-OR⁷, —C(O)R⁷, —C(O)N(R⁷)₂, —CN, or —N(R⁷)₂. Other embodiments of interest to the present inventors are those compounds additionally wherein R³ is hydrogen.

In any of the embodiments described above, R³ is hydrogen, C₁₋₆alkyl, or —(C(R⁶)2)ₜ-(C₃-₆cycloalkyl), wherein said alkyl or cycloalkyl moiety may be optionally independently substituted with one to three fluorines; and R⁴ is a —(C(R⁶)₂)ₜ-(4- to 10-membered heterocycloalkyl); wherein said heterocycloalkyl moiety may be optionally substituted with one to three R⁶. In any of the embodiments described above, R⁴ is —(C(R⁶)₂)ₜ-(4- to 10-membered heterocycloalkyl); wherein said heterocycloalkyl moiety may be optionally substituted with one R⁶, and R⁶ is selected from C₁₋₆alkyl, —CF₃, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —CN, or —N(R⁷)₂. In any of the embodiments described above, R⁴ is —(C(R⁶)₂)ₜ-(4- to 10-membered heterocycloalkyl); wherein said heterocycloalkyl moiety may be optionally substituted with one R⁶, and R⁶ is halogen, —CF₃, —CN, C₃₋₆cycloalkyl or C₆₋₁₀aryl. In any of the embodiments described above, R⁴ is —(C(R⁶)₂)ₜ-(4- to 10-membered heterocycloalkyl); wherein said heterocycloalkyl moiety may be optionally substituted with two R⁶ and each R⁶ is independently selected from C₁₋₆alkyl, C₂₋₆alkenyl, —CF₃, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —CN, or —N(R⁷)₂. In any of the embodiments described above, R⁴ is —(C(R⁶)₂)ₜ-(4- to 10-membered heterocycloalkyl); wherein said heterocycloalkyl moiety may be optionally substituted with three R⁶, and each R⁶ is independently selected from C₁₋₆alkyl, C₂₋₆alkenyl, —CF₃, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —CN, or —N(R⁷)₂. Other embodiments of interest to the present inventors are those compounds additionally wherein R³ is hydrogen.

In any of the embodiments described above, R³ is hydrogen, C₁₋₆alkyl, or —(C(R⁶)₂)ₜ-(C₃₋₆cycloalkyl); wherein said alkyl or cycloalkyl moiety may be optionally independently substituted with one to three fluorines; and R⁴ is —(C(R⁶)₂)ₜ-(C₆₋₁₀aryl); wherein said aryl moiety may be optionally substituted with one to three substituents selected from R⁶. In another embodiment of the invention, R⁴ is —(C(R⁶)2)ₜ-(C₆₋₁₀aryl); wherein said aryl moiety may be optionally substituted with one R⁶, and R⁶ is selected from C₁₋₆alkyl, C₂₋₆alkenyl, —CF₃, —CN, halogen, —(C(R⁹)₂)ₘ-(C₃₋₅cycloalkyl), —(C(R)₂)ₘ-(4- to 10-membered heterocycloalkyl), —(C(R)₂)ₘ-(C₆₋₁₀aryl), or —(C(R)₂)ₘ-(5- to 10-membered heteroaryl), —(C(R⁹)₂)ₘ-OR⁷, —C(O)R⁷, —C(O)N(R′)₂, —CN, or —N(R⁷)₂. In another embodiment of the invention, R⁴ is —(C(R⁶)₂)ₜ-(C₆₋₁₀aryl); wherein said aryl moiety may be optionally substituted with one R⁶ and R⁶ is halogen or —CN. In one embodiment of the invention R⁴ is —(C(R⁶)₂)ₜ-(C₆₋₁₀aryl); wherein said aryl moiety may be optionally substituted with two R⁶, and each R⁶ is independently selected from C₁₋₆alkyl, C₂₋₆alkenyl, —CF₃, —CN, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R)₂)ₘ-OR⁷, —C(O)R⁷, —C(O)N(R⁷)₂, —CN, or —N(R⁷)₂. In one embodiment of the invention is —(C(R⁶)₂)ₜ-(C₆₋₁₀aryl); wherein said aryl moiety may be optionally substituted with three R⁶, and each R⁶ is independently selected from C₁₋₆alkyl, C₂₋₆alkenyl, —CF₃, —CN, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)², or —N(R⁷)₂. Other embodiments of interest to the present inventors are those compounds additionally wherein is hydrogen.

In any of the embodiments described above, R³ is hydrogen, C₁₋₆alkyl, or —(C(R⁶)₂)ₜ-(C₃₋₆cycloalkyl); wherein said alkyl or cycloalkyl moiety may be optionally independently substituted with one to three fluorines; and R⁴ is —(C(R⁸)₂)ₜ-(5- to 10-membered heteroaryl); wherein said heteroaryl moiety may be optionally substituted with one to three substituents independently selected from R⁶. In one embodiment of the invention R⁴ i —(C(R⁶)₂)ₜ-(5- to 10-membered heteroaryl); wherein said heteroaryl moiety may be optionally substituted with one R⁶, and R⁶ is selected from C₁₋₆alkyl, C₂₋₆alkenyl, —CF₃, —CN, halogen, —(C(R⁹)₂)ₘ-(C₃₋₆cycloalkyl), —(C(R⁹)₂)ₘ-(4- to 10-membered heterocycloalkyl), —(C(R²)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, or —N(R⁷)₂. In one embodiment of the invention R⁴ is —(C(R⁶)₂)ₜ-(5- to 10-membered heteroaryl); wherein said heteroaryl moiety may be optionally independently substituted with one R⁶, and R⁶ is halogen or —CN. In one embodiment of the invention R⁴ is —(C(R⁶)₂)ₜ-(5-to 10-membered heteroaryl); wherein said heteroaryl moiety may be optionally independently substituted with two R⁶, and each R⁶ is independently selected from C₁₋₆alkyl, C₂₋₆alkenyl, —CF₃, —CN, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), ^{or —(C(R}⁹)₂)ₘ-(5- to 10-membered heteroaryl), —(C(R⁹)₂)ₘ-OR⁷, —C(O)R⁷, —C(O)N(R⁷)₂, or —N(R⁷)₂. In one embodiment of the invention R⁴ is —(C(R⁶)₂)ₜ-(5- to 10-membered heteroaryl); wherein said heteroaryl moiety may be optionally independently substituted with three R⁶ and each R⁶ is independently selected from C₁₋₆alkyl, C₂₋₆alkenyl, —CF₃, cyano, halogen, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, or —N(R⁷)₂. Other embodiments of interest to the present inventors are those compounds additionally wherein R³ is hydrogen.

In another embodiment of the invention R¹ is C₁₋₆alkyl. In another embodiment of this invention, R¹ is methyl.

In another embodiment of the invention R² is halogen.

In another embodiment of the invention R² is —OR⁵. In one embodiment of the invention, R² is —OR⁵; wherein R⁵ is hydrogen or C₁₋₆alkyl. In one embodiment of the invention R² is —OR⁵; wherein R⁵ is hydrogen. In another embodiment of the invention R² is —OR⁵; wherein R⁵ is C₁₋₆alkyl. In an example of this embodiment, R⁵ is methyl.

In one embodiment of the invention, R³ is hydrogen, C₁₋₆alkyl, or —(C(R⁶)₂)ₜ-(C₃₋₆cycloalkyl); wherein said alkyl or cycloalkyl moiety may be optionally independently substituted with one to three fluorines.

In one embodiment of the invention R³ is hydrogen.

In one embodiment of the invention R³ is C₁₋₆alkyl. In another embodiment of the invention, R³ is methyl.

It is understood that descriptions of any one substituent, such as R¹, may be combined with descriptions of any other substituents, such as R², such that each and every combination of the first substituent and the second substituent is provided herein the same as if each combination were specifically and individually listed. For example, in one variation, R¹ is taken together with R² to provide an embodiment wherein R¹ is methyl and R² is halogen.

It will be understood that the compounds of formula I, and pharmaceutically acceptable salts thereof, also include hydrates, solvates and polymorphs of said compounds of formula I, and pharmaceutically acceptable salts thereof, as discussed below.

In one embodiment, the invention also relates to each of the individual compounds described as Examples 1 to 56 in the Examples section of the subject application, (including the free bases or pharmaceutically acceptable salts thereof).

In another embodiment the invention relates to a compound selected from the group consisting of:

5-(4-methyl-1H-imidazol-1-yl)-2-({(3S)-3-[2-(trifluoromethyl)phenoxy]pyrrolidin-1-yl}carbonyl)pyridine;

2-(4-methyl-1H-imidazol-1-yl)-5-({(3S)-3-[2-(trifluoromethyl)phenoxy]pyrrolidin-1-yl}carbonyl)pyrimidine;

2-(4-methyl-1H-imidazol-1-yl)-5-({(3S)-3-[2-(trifluoromethyl)phenoxy]pyrrolidin-1-yl}carbonyl)pyrazine;

5-(4-methyl-1H-imidazol-1-yl)-2-({(3S)-3-[2-(trifluoromethyl)phenoxy]pyrrolidin-1-yl}carbonyl)pyrimidine;

3-(4-methyl-1H-imidazol-1-yl)-6-({(3S)-3-[2-(trifluoromethyl)phenoxy]pyrrolidin-1-yl}carbonyl)pyridazine;

N-(2,5-dimethylbenzyl)-5-(4-methyl-1H-imidazol-1-yl)pyridine-2-carboxamide;

N-(2,5-dimethylbenzyl)-2-(4-methyl-1H-imidazol-1-yl)pyrimidine-5-carboxamide;

N-(2,5-dimethylbenzyl)-5-(4-methyl-1H-imidazol-1-yl)pyrazine-2-carboxamide;

N-(2,5-dimethylbenzyl)-5-(4-methyl-1H-imidazol-1-yl)pyrimidine-2-carboxamide;

N-(2,5-dimethylbenzyl)-6-(4-methyl-1H-imidazol-1-yl)pyridazine-3-carboxamide;

N-{[1-(4-fluorophenyl)cyclopropyl]methyl}-5-(4-methyl-1H-imidazol-1-yl)pyridine-2-carboxamide;

N-{[1-(4-fluorophenyl)cyclopropyl]methyl}-2-(4-methyl-1H-imidazol-1-yl)pyrimidine-5-carboxamide;

N-{[1-(4-fluorophenyl)cyclopropyl]methyl}-5-(4-methyl-1H-imidazol-1-yl)pyrazine-2-carboxamide;

N-{[1-(4-fluorophenyl)cyclopropyl]methyl}-5-(4-methyl-1H-imidazol-1-yl)pyrimidine-2-carboxamide;

N-{[1-(4-fluorophenyl)cyclopropyl]methyl}-6-(4-methyl-1H-imidazol-1-yl)pyridazine-3-carboxamide;

N-[(3R)-5-chloro-2,3-dihydro-1-benzofuran-3-yl]-6-methoxy-5-(4-methyl-1H-imidazol-1-yl)pyridine-2-carboxamide;

N-[(3S)-5-chloro-2,3-dihydro-1-benzofuran-3-yl]-6-methoxy-5-(4-methyl-1H-imidazol-1-yl)pyridine-2-carboxamide;

N-[(3R)-7-chloro-2,3-dihydro-1-benzofuran-3-yl]-6-methoxy-5-(4-methyl-1H-imidazol-1-yl)pyridine-2-carboxamide;

N-[(3S)-7-chloro-2,3-dihydro-1-benzofuran-3-yl]-6-methoxy-5-(4-methyl-1H-imidazol-1-yl)pyridine-2-carboxamide;

N-[(3R)-6-chloro-2,3-dihydro-1-benzofuran-3-yl]-6-methoxy-5-(4-methyl-1H-imidazol-1-yl)pyridine-2-carboxamide;

N-[(3S)-6-chloro-2,3-dihydro-1-benzofuran-3-yl]-6-methoxy-5-(4-methyl-1H-imidazol-1-yl)pyridine-2-carboxamide;

N-[(3R)-4,6-dichloro-2,3-dihydro-1-benzofuran-3-yl]-6-methoxy-5-(4-methyl-1H-imidazol-1-yl)pyridine-2-carboxamide;

N-[(3S)-4,6-dichloro-2,3-dihydro-1-benzofuran-3-yl]-6-methoxy-5-(4-methyl-1H-imidazol-1-yl)pyridine-2-carboxamide;

N-[(3R)-5,7-dichloro-2,3-dihydro-1-benzofuran-3-yl]-6-methoxy-5-(4-methyl-1H-imidazol-1-yl)pyridine-2-carboxamide;

N-[(3S)-5,7-dichloro-2,3-dihydro-1-benzofuran-3-yl]-6-methoxy-5-(4-methyl-1H-imidazol-1-yl)pyridine-2-carboxamide;

N-[(3R)-5-cyclopropyl-6-fluoro-2,3-dihydro-1-benzofuran-3-yl]-6-methoxy-5-(4-methyl-1H-imidazol-1-yl)pyridine-2-carboxamide;

N-[(3S)-5-cyclopropyl-6-fluoro-2,3-dihydro-1-benzofuran-3-yl]-6-methoxy-5-(4-methyl-1H-imidazol-1-yl)pyridine-2-carboxamide;

N-[(3R)-6-cyclopropyl-5-fluoro-2,3-dihydro-1-benzofuran-3-yl]-6-methoxy-5-(4-methyl-1H-imidazol-1-yl)pyridine-2-carboxamide;

N-[(3S)-6-cyclopropyl-5-fluoro-2,3-dihydro-1-benzofuran-3-yl]-6-methoxy-5-(4-methyl-1H-imidazol-1-yl)pyridine-2-carboxamide;

N-[(3R)-5-cyclopropyl-2,3-dihydro-1-benzofuran-3-yl]-6-methoxy-5-(4-methyl-1H-imidazol-1-yl)pyridine-2-carboxamide;

N-[(3S)-5-cyclopropyl-2,3-dihydro-1-benzofuran-3-yl]-6-methoxy-5-(4-methyl-1H-imidazol-1-yl)pyridine-2-carboxamide;

6-methoxy-5-(4-methyl-1H-imidazol-1-yl)-N-[(3R)-5-(trifluoromethyl)-2,3-dihydro-1-benzofuran-3-yl]pyridine-2-carboxamide;

6-methoxy-5-(4-methyl-1H-imidazol-1-yl)-N-[(3S)-5-(trifluoromethyl)-2,3-dihydro-1-benzofuran-3-yl]pyridine-2-carboxamide;

6-methoxy-5-(4-methyl-1H-imidazol-1-yl)-N-[(3R)-6-(trifluoromethyl)-2,3-dihydro-1-benzofuran-3-yl]pyridine-2-carboxamide;

6-methoxy-5-(4-methyl-1H-imidazol-1-yl)-N-[(3S)-6-(trifluoromethyl)-2,3-dihydro-1-benzofuran-3-yl]pyridine-2-carboxamide;

N-[(3R)-5-isopropyl-2,3-dihydro-1-benzofuran-3-yl]-6-methoxy-5-(4-methyl-1H-imidazol-1-yl)pyridine-2-carboxamide;

N-[(3S)-5-isopropyl-2,3-dihydro-1-benzofuran-3-yl]-6-methoxy-5-(4-methyl-1H-imidazol-1-yl)pyridine-2-carboxamide;

N-[(3R)-6-ethoxy-5-isopropyl-2,3-dihydro-1-benzofuran-3-yl]-6-methoxy-5-(4-methyl-1H-imidazol-1-yl)pyridine-2-carboxamide;

N-[(3S)-6-ethoxy-5-isopropyl-2,3-dihydro-1-benzofuran-3-yl]-6-methoxy-5-(4-methyl-1H-imidazol-1-yl)pyridine-2-carboxamide;

N-[(3R)-5-chloro-6-ethoxy-2,3-dihydro-1-benzofuran-3-yl]-6-methoxy-5-(4-methyl-1H-imidazol-1-yl)pyridine-2-carboxamide;

N-[(3S)-5-chloro-6-ethoxy-2,3-dihydro-1-benzofuran-3-yl]-6-methoxy-5-(4-methyl-1H-imidazol-1-yl)pyridine-2-carboxamide;

6-methoxy-5-(4-methyl-1H-imidazol-1-yl)-N-[(3R)-5-phenyl-2,3-dihydro-1-benzofuran-3-yl]pyridine-2-carboxamide;

6-methoxy-5-(4-methyl-1H-imidazol-1-yl)-N-[(3S)-5-phenyl-2,3-dihydro-1-benzofuran-3-yl]pyridine-2-carboxamide;

6-methoxy-N-[(3R)-5-methyl-2,3-dihydro-1-benzofuran-3-yl]-5-(4-methyl-1H-imidazol-1-yl)pyridine-2-carboxamide;

6-methoxy-N-[(3S)-5-methyl-2,3-dihydro-1-benzofuran-3-yl]-5-(4-methyl-1H-imidazol-1-yl)pyridine-2-carboxamide;

N-[(3R)-5,6-dimethyl-2,3-dihydro-1-benzofuran-3-yl]-6-methoxy-5-(4-methyl-1H-imidazol-1-yl)pyridine-2-carboxamide;

N-[(3S)-5,6-dimethyl-2,3-dihydro-1-benzofuran-3-yl]-6-methoxy-5-(4-methyl-1H-imidazol-1-yl)pyridine-2-carboxamide;

6-methoxy-5-(4-methyl-1H-imidazol-1-yl)-N-[(3R)-5-phenoxy-2,3-dihydro-1-benzofuran-3-yl]pyridine-2-carboxamide;

6-methoxy-5-(4-methyl-1H-imidazol-1-yl)-N-[(3S)-5-phenoxy-2,3-dihydro-1-benzofuran-3-yl]pyridine-2-carboxamide;

6-methoxy-5-(4-methyl-1H-imidazol-1-yl)-N-[(3R)-6-phenoxy-2,3-dihydro-1-benzofuran-3-yl]pyridine-2-carboxamide;

6-methoxy-5-(4-methyl-1H-imidazol-1-yl)-N-[(3S)-6-phenoxy-2,3-dihydro-1-benzofuran-3-yl]pyridine-2-carboxamide;

2-methoxy-3-(4-methyl-1H-imidazol-1-yl)-6-({3-[(1-naphthyloxy)methyl]azetidin-1-yl}carbonyl)pyridine;

N-[5-chloro-4-fluoro-2,3-dihydro-1-benzofuran-3-yl]-6-methoxy-5-(4-methyl-1H-imidazol-1-yl)pyridine-2-carboxamide;

N-[5-chloro-6-methyl-2,3-dihydro-1-benzofuran-3-yl]-6-methoxy-5-(4-methyl-1H-imidazol-1-yl)pyridine-2-carboxamide;

N-(5-chloro-4-methyl-2,3-dihydro-1-benzofuran-3-yl)-6-methoxy-5-(4-methyl-1H-imidazol-1-yl)pyridine-2-carboxamide;

N-(5-chloro-7-methyl-2,3-dihydro-1-benzofuran-3-yl)-6-methoxy-5-(4-methyl-1H-imidazol-1-yl)pyridine-2-carboxamide;

N-[(3R)-5-chloro-2,3-dihydro-1-benzofuran-3-yl]-6-methoxy-5-(4-methyl-1H-imidazol-1-yl)pyridine-2-carboxamide;

N-[4-fluoro-5-(trifluoromethyl)-2,3-dihydro-1-benzofuran-3-yl]-6-methoxy-5-(4-methyl-1H-imidazol-1-yl)pyridine-2-carboxamide;

N-[5-chloro-6-fluoro-2,3-dihydro-1-benzofuran-3-yl]-6-methoxy-5-(4-methyl-1H-imidazol-1-yl)pyridine-2-carboxamide;

6-methoxy-5-(4-methyl-1H-imidazol-1-yl)-N-[5-(trifluoromethyl)-2,3-dihydro-1-benzofuran-3-yl]pyridine-2-carboxamide

and the pharmaceutically acceptable salts of each of the foregoing.

In another embodiment of the present invention, compounds of formula I are optionally used in combination with another active agent. Such an active agent may be, for example, an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

The invention is also directed to a pharmaceutical composition comprising a compound of formula I, and a pharmaceutically acceptable carrier. The composition may be, for example, a composition for treating a condition selected from the group consisting of neurological and psychiatric disorders, including but not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering an effective amount of compound of formula I or pharmaceutically acceptable salt thereof, and a pharmaceutically acceptable carrier. The composition optionally further comprises an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

### Definitions

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, isoamyl, hexyl and the like. In some instances, the number of carbon atoms in a hydrocarbyl substituent (i.e., alkyl, alkenyl, cycloalkyl, aryl, etc.) is indicated by the prefix “C_{x-y},” wherein x is the minimum and y is the maximum number of carbon atoms in the substituent. Thus, for example, “C₁₋₆alkyl” refers to an alkyl substituent containing from 1 to 6 carbon atoms.

“Alkenyl” refers to an aliphatic hydrocarbon having at least one carbon-carbon double bond, including straight chain, branched chain or cyclic groups having at least one carbon-carbon double bond. Preferably, it is a medium size alkenyl having 2 to 6 carbon atoms. For example, as used herein, the term “C₂₋₆alkenyl” means straight or branched chain unsaturated radicals of 2 to 6 carbon atoms, including, but not limited to ethenyl, 1-propenyl, 2-propenyl(allyl), isopropenyl, 2-methyl-1-propenyl, 1-butenyl, 2-butenyl, and the like; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or C₁-C₆alkyl. When the compounds of the invention contain a C₂-₆alkenyl group, the compound may exist as the pure E (entgegen) form, the pure Z (zusammen) form, or any mixture thereof.

“Alkylidene” refers to a divalent group formed from an alkane by removal of two hydrogen atoms from the same carbon atom, the free valencies of which are part of a double bond.

“Alkynyl” refers to an aliphatic hydrocarbon having at least one carbon-carbon triple bond, including straight chain, branched chain or cyclic groups having at least one carbon-carbon triple bond. Preferably, it is a lower alkynyl having 2 to 6 carbon atoms. For example, as used herein, the term “C₂₋₆alkynyl” is used herein to mean a straight or branched hydrocarbon chain alkynyl radical as defined above having 2 to 6 carbon atoms and one triple bond.

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5- to 10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or 5-10-membered heteroaromatic ring may be optionally substituted with halogen, C₁₋₆alkyl, C₃₋₁₀cycloalkyl, or ═O.

A cycloalkyl may be a single ring, which typically contains from 3 to 6 ring atoms. Examples include cyclopropyl, cyclobutyl, cyclopentyl, and cyclohexyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄₋₁₀ carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄₋₁₀ carbocyclic or 4- to 10-membered heterocyclic ring may be optionally substituted with halogen, C₁₋₆alkyl, C₃₋₁₀cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of atoms in a cyclic substituent containing one or more heteroatoms (i.e., heteroaryl or heterocycloalkyl) is indicated by the prefix “X-Y-membered”, wherein wherein x is the minimum and y is the maximum number of atoms forming the cyclic moiety of the substituent. Thus, for example, 5- to 8-membered heterocycloalkyl refers to a heterocycloalkyl containing from 5 to 8 atoms, including one or more heteroatoms, in the cyclic moiety of the heterocycloalkyl.

The term “hydrogen” refers to a hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents are attached include, for example, alcohols, enols and phenol.

The term “cyano” (also referred to as “nitrile”) means —CN, which also may be depicted:

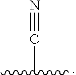

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is fluorine. In another embodiment, the halogen is bromine.

The term “heterocycloalkyl” refers to a substituent obtained by removing a hydrogen from a saturated or partially saturated ring structure containing a total of 4 to 14 ring atoms, wherein at least one of the ring atoms is a heteroatom selected from oxygen, nitrogen, or sulfur. For example, as used herein, the term “4- to 10-membered heterocycloalkyl” means the substituent is a single ring with 4 to 10 total members. A heterocycloalkyl alternatively may comprise 2 or 3 rings fused together, wherein at least one such ring contains a heteroatom as a ring atom (i.e., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆₋₁₀aromatic ring or to a 5- to 10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one or more substituents, the one or more substituents, unless otherwise specified, are each bound to a heteroatom of the heterocycloalkyl group or to a carbon atom of the heterocycloalkyl group. The fused C₆-C₁₀ aromatic ring or 5- to 10-membered heteroaromatic ring may be optionally substituted with halogen, C₁₋₆alkyl, C₃₋₁₀cycloalkyl, C₁₋₆alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include but are not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls and heterocycloalkyls include but are not limited to furanyl, dihydrofuranyl, tetrahydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, isoxazolinyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), morpholinyl, azepinyl, oxepinyl, thiepinyl, and diazepinyl.

Examples of 2-fused-ring heteroaryls include but are not limited to indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include but are not limited to 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include but are not limited to benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄₋₁₀ carbocyclic ring, such as a C₅ or a C₆ carbocyclic ring, or to a 4-10-membered heterocyclic ring, wherein a group having such a fused heteroaryl group as a substituent is bound to an aromatic carbon of the heteroaryl group or to a heteroatom of the heteroaryl group. When such a fused heteroaryl group is substituted with one or more substituents, the one or more substitutents, unless otherwise specified, are each bound to an aromatic carbon of the heteroaryl group or to a heteroatom of the heteroaryl group. The fused C₄₋₁₀ carbocyclic or 4-10-membered heterocyclic ring may be optionally substituted with halogen, C₁₋₆ alkyl, C₃₋₁₀ cycloalkyl, or ═O.

Additional examples of heteroaryls and heterocycloalkyls include but are not limited to: 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-a]pyridine, benzothianyl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached).

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″, wherein R′ and R″ together with the nitrogen atom to which they are attached may form a heterocyclic ring comprising 1 or 2 heteroatoms independently selected from oxygen, nitrogen, or sulfur, wherein said heterocycloalkyl moiety may be optionally substituted. The heterocyclic ring formed from R′ and R″ together with the nitrogen atom to which they are attached may be partially or fully saturated, or aromatic. In one embodiment, the heterocyclic ring consists of 4 to 10 atoms. In another embodiment, the heterocyclic ring is selected from the group consisting of piperidinyl, morpholinyl, azetidinyl, pyrrolyl, imidazolyl, pyrazolyl, triazolyl, tetrazolyl, isoxazolyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁₋₆- prefix on C₁₋₆alkylcycloalkyl means that the alkyl moiety of the alkylcycloalkyl contains from 1 to 6 carbon atoms; the C₁₋₆- prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution only occurs on the alkyl moiety, the substituent would be described as “alkoxyhaloalkyl.” If the halogen substitution occurs on both the alkyl moiety and the alkoxy moiety, the substituent would be described as “haloalkoxyhaloalkyl.”

If substituents are described as being “independently selected” from a group, each substituent is selected independent of the other(s). Each substituent therefore may be identical to or different from the other substituent(s).

As used herein the term “Formula I” may be hereinafter referred to as a “compound(s) of the invention.” Such terms are also defined to include all forms of the compound of Formula I, including hydrates, solvates, isomers, crystalline and non-crystalline forms, isomorphs, polymorphs, and metabolites thereof. For example, the compounds of Formula I, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of Formula I may exist as clathrates or other complexes. Included within the scope of the invention are complexes such as clathrates, drug-host inclusion complexes wherein, in contrast to the aforementioned solvates, the drug and host are present in stoichiometric or non-stoichiometric amounts. Also included are complexes of Formula I containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

The compounds of Formula I may have asymmetric carbon atoms. The carbon-carbon bonds of the compounds of Formula I may be depicted herein using a solid line (——) a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I can exist as enantiomers and diastereomers or as racemates and mixtures thereof. The use of a solid line to depict bonds to one or more asymmetric carbon atoms in a compound of Formula I and the use of a solid or dotted wedge to depict bonds to other asymmetric carbon atoms in the same compound is meant to indicate that a mixture of diastereomers is present.

Stereoisomers of Formula I include cis and trans isomers, optical isomers such as R and S enantiomers, diastereomers, geometric isomers, rotational isomers, conformational isomers, and tautomers of the compounds of Formula I, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I may exhibit the phenomena of tautomerism and structural isomerism. For example, the compounds of Formula I may exist in several tautomeric forms, including the enol and imine forms, and the keto and enamine forms, and geometric isomers and mixtures thereof. All such tautomeric forms are included within the scope of compounds of Formula I. Tautomers exist as mixtures of a tautomeric set in solution. In solid form, usually one tautomer predominates. Even though one tautomer may be described, the present invention includes all tautomers of the compounds of Formula I.

The present invention also includes isotopically-labeled compounds, which are identical to those recited in Formula I above, but for the fact that one or more atoms are replaced by an atom having an atomic mass or mass number different from the atomic mass or mass number usually found in nature. Examples of isotopes that may be incorporated into compounds of Formula I include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³²P, ³⁵S, ¹⁸F, and ³⁶Cl. Certain isotopically-labeled compounds of Formula I, for example those into which radioactive isotopes such as ³H and ¹⁴H are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of Formula I may generally be prepared by carrying out the procedures disclosed in the Schemes and/or in the Examples and Preparations below, by substituting an isotopically-labeled reagent for a non-isotopically-labeled reagent.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include but are not limited to aliphatic, cycloaliphatic, aromatic, araliphatic, heterocyclic, carboxylic, and sulfonic classes of organic acids.

Specific examples of suitable organic acids include but are not limited to acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts. The term “therapeutically effective amount” as used herein refers to that amount of the compound being administered which will relieve to some extent one or more of the symptoms of the disorder being treated.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear. The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, the total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the invention comprises the use of one or more compounds of the invention for the preparation of a medicament for the treatment of the conditions recited herein.

For the treatment of the conditions referred to above, the compounds of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Formulations suitable for topical administration to the eye include, for example, eye drops wherein the compound of this invention is dissolved or suspended in a suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

In another embodiment, the invention relates to the novel intermediates useful for preparing the compounds of the invention.

### General Synthetic Schemes

The compounds of formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-XII (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They do not constrain the scope of the invention in any way.

### Experimental Procedures and Working Examples

The following illustrate the synthesis of various compounds of the present invention. Additional compounds within the scope of this invention may be prepared using the methods illustrated in these Examples, either alone or in combination with techniques generally known in the art.

It will be understood that the intermediate compounds of the invention depicted above are not limited to the particular enantiomer shown, but also include all stereoisomers and mixtures thereof. It will also be understood that compounds of Formula I can include intermediates of compounds of Formula I.

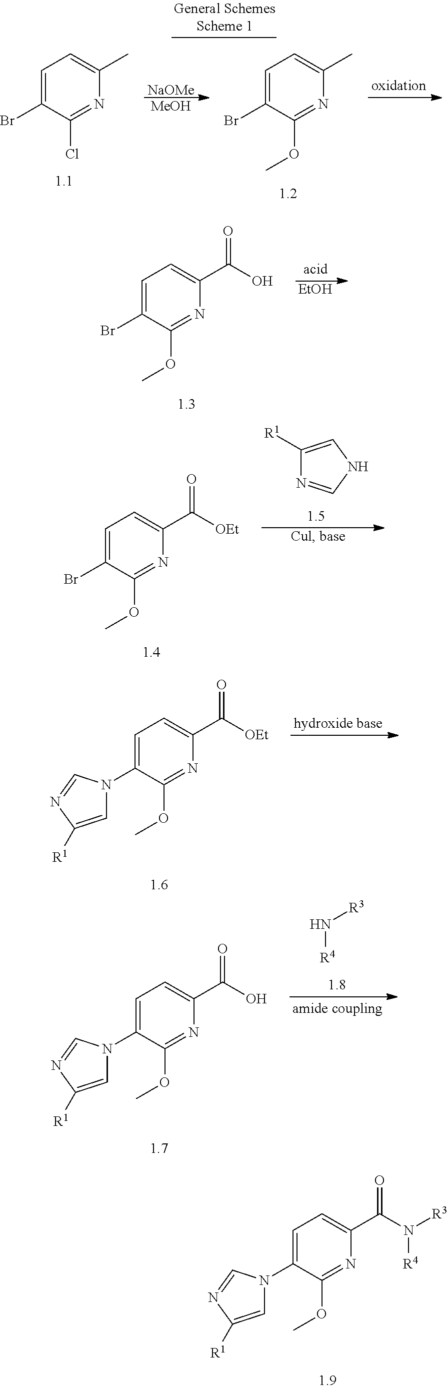

Scheme 1 illustrates a method for preparing compounds depicted by formula 1.9. This method commences with the addition of sodium methoxide to 3-bromo-2-chloro-6-methylpyridine (1.1) to furnish the corresponding methoxy-substituted pyridyl intermediate of formula 1.2. The methyl substituent of intermediate 1.2 may then be oxidized to the carboxylic acid 1.3 under a variety of conditions including oxidation with selenium dioxide in a solvent such as Dowtherm. The compound of formula 1.3 is then converted to an ester such as ethyl ester 1.4 by refluxing in ethanol in the presence of a suitable acid such as p-toluenesulfonic acid, H₂SO₄ or HCl. The compound of formula 1.4 is then coupled with imidazole 1.5 by heating the mixture in the presence of copper iodide and a suitable base such as cesium carbonate in a solvent such as DMF. The resulting ester of formula 1.6 is then hydrolyzed by treating with aqueous base such as KOH or LiOH in a solvent such as MeOH or THF. The resulting acid of formula 1.7 is then subjected to an amide bond coupling with an amine of the formula 1.8 using one of the many amide bond coupling strategies known to those skilled in the art. For example, this reaction may be performed using HATU [O-(7-azabenzotriazol-1-yl)-N,N,N′,N′-tetramethyluronium hexafluorophosphate] or another suitable coupling reagent such as EDCI [N-[3-(dimethylamino)propyl]-N′-ethylcarbodiimide hydrochloride] and HOBT [1H-benzotriazol-1-ol] in the presence of a base such as diisopropylethylamine to form the corresponding amide of the formula 1.9.

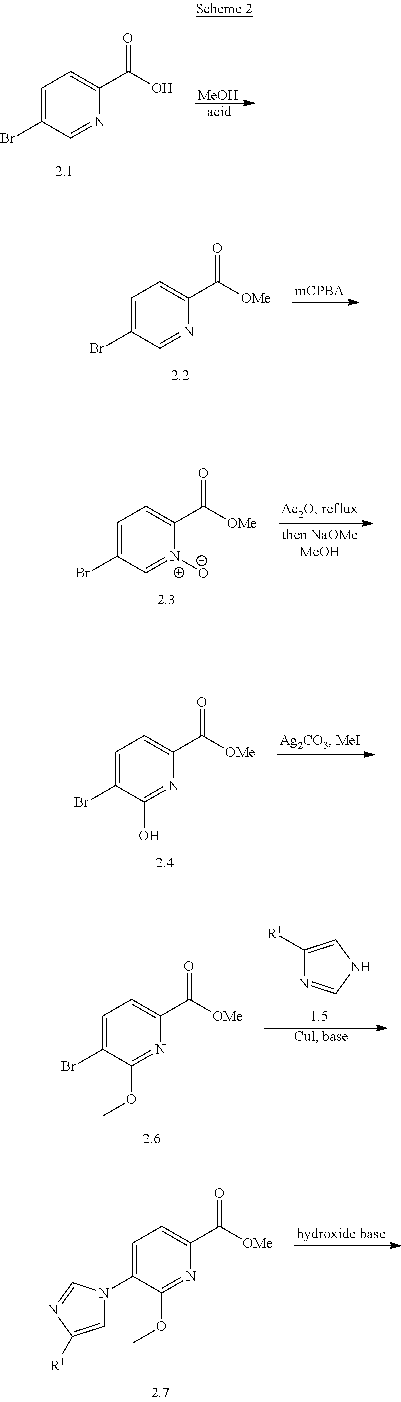

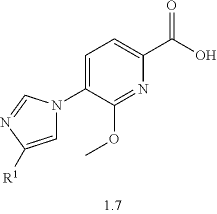

Scheme 2 illustrates an alternative method for the preparation of intermediate 1.7. The acid of formula 2.1 is converted to the methyl ester of formula 2.2 by refluxing in MeOH in the presence of an acid such as H₂SO₄. The intermediate of formula 2.2 is then oxidized by mCPBA in a solvent such as chloroform to afford the corresponding N-oxide of formula 2.3. This compound is then refluxed in acetic anhydride followed by exposure to NaOMe in MeOH. The resulting 2-hydroxypyridyl intermediate of formula 2.4 may then be alkylated with Mel in the presence of Ag₂CO₃ in a solvent such as chloroform to furnish the 2-methoxypyridyl intermediate of formula 2.6. The compound of formula 2.6 is coupled with imidazole 1.5 by heating the mixture in the presence of copper iodide and a suitable base such as cesium carbonate in a solvent such as DMF. The resulting ester of formula 2.7 is then hydrolyzed by treating with aqueous base such as KOH or LiOH in a solvent such as MeOH or THF.

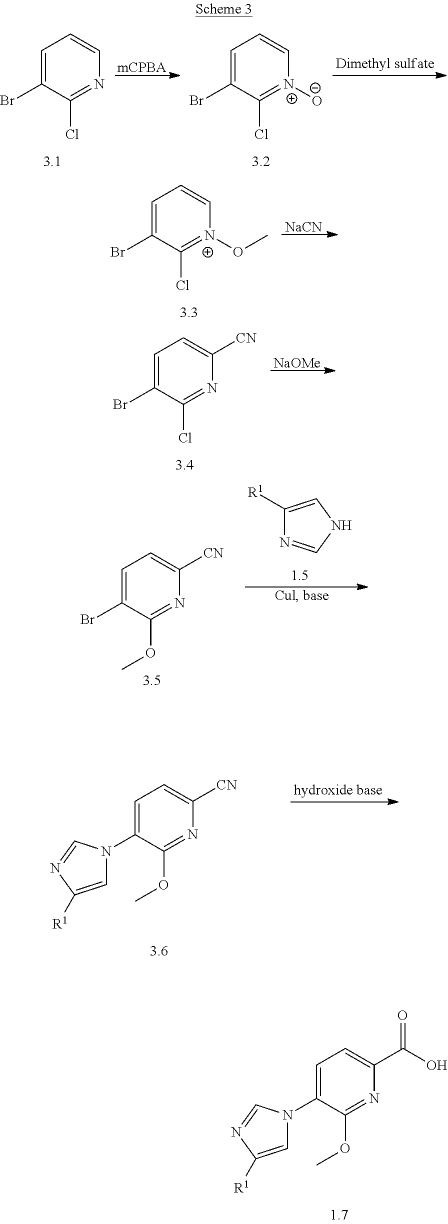

Scheme 3 illustrates an alternative method for the preparation of intermediate 1.7. This method commences with the oxidation of 3-bromo-2-chloropyridine (3.1) using mCPBA in a solvent such as DCE. The resulting N-oxide of formula 3.2 is then alkylated with dimethyl sulfate in a solvent such as CH₃CN to provide intermediate 3.3, which is treated with aqueous NaCN to give the 2-cyanopyridyl derivative of formula 3.4. The compound of formula 3.4 is reacted with sodium methoxide to afford a 2-methoxypyridyl derivative of formula 3.5. This intermediate is coupled with imidazole 1.5 by heating the mixture in the presence of copper iodide and a suitable base such as cesium carbonate in a solvent such as DMF. The resulting nitrile of formula 3.6 is then hydrolyzed by treating with aqueous base such as NaOH, LiOH or KOH to afford the acid of formula 1.7.

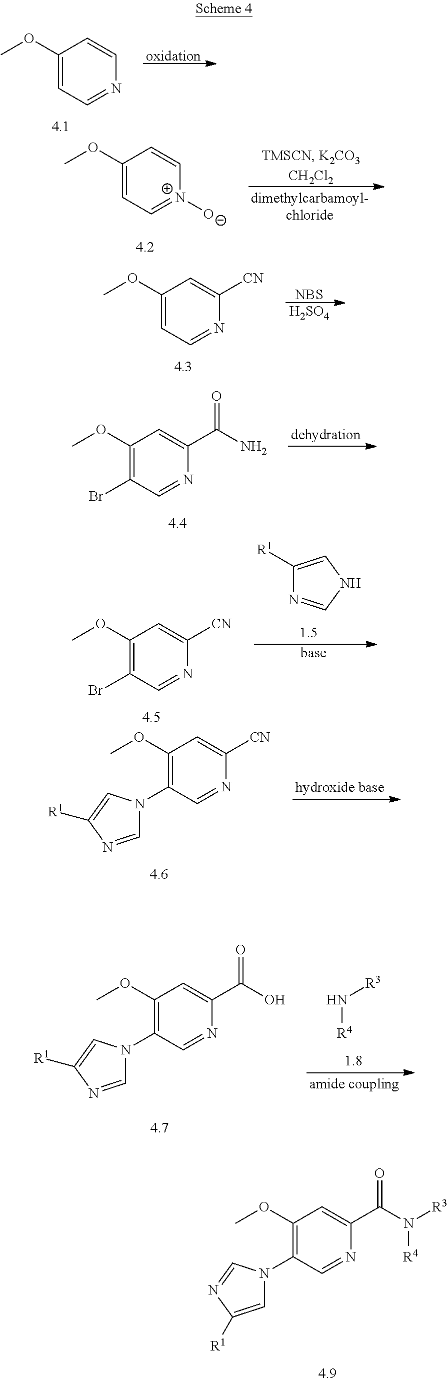

Scheme 4 illustrates a method for preparing amide derivatives depicted by formula 4.9. 4-Methoxypyridine (4.1) is oxidized to the corresponding N-oxide 4.2 using an oxidizing agent such as H₂O₂ in AcOH. The intermediate of formula 4.2 is then treated with dimethylcarbamoyl chloride and TMSCN to afford 4-methoxypyridine-2-carbonitrile (4.3). Bromination of 4.3 with NBS in H₂SO₄ furnishes an intermediate of formula 4.4, which is converted to 4.5 by exposure to a suitable dehydrating reagent system such as oxalyl chloride and pyridine in DMF. The compound of formula 4.5 is then coupled with imidazole 1.5 by heating the mixture in the presence of 18-crown-6 and a base such as K₂CO₃ in a solvent such as CH₃CN. Alternatively the coupling may be carried out as described in Schemes 1-3 by using copper iodide and a suitable base such as cesium carbonate in a solvent such as DMF. The resulting nitrile of formula 4.6 is then hydrolyzed by treating with aqueous base such as KOH or LiOH in a solvent such as MeOH or THF. The resulting acid of formula 4.7 is then subjected to an amide bond coupling with an amine of the formula 1.8 using HATU or another suitable coupling reagent such as EDCI and HOBT in the presence of a base such as diisopropylethylamine to form the corresponding amide of the formula 4.9.

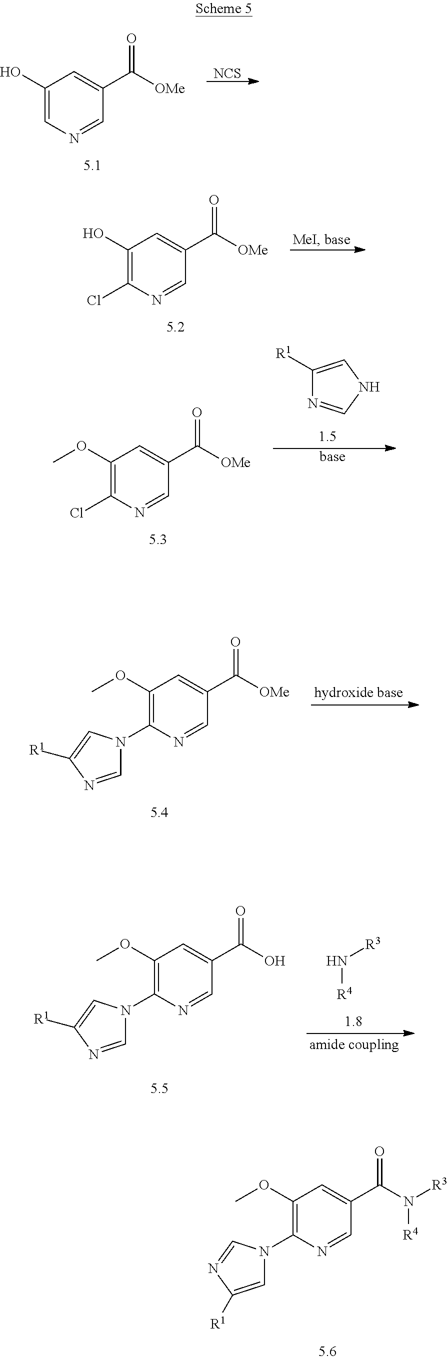

Scheme 5 illustrates a method for the synthesis of compounds of formula 5.6. Methyl 5-hydroxynicotinate (5.1) is chlorinated using NCS in DMF to afford an intermediate of formula 5.2. The hydroxyl substituent of 5.2 is then alkylated with methyl iodide and a base such as K₂O0₃ in a solvent such as acetone. The resulting intermediate of formula 5.3 may then be coupled with imidazole 1.5 by heating the mixture in the presence of a base such as CsF in a solvent such as DMSO, DMAC or DMF. The resulting ester of formula 5.4 is then hydrolyzed by treating with aqueous base such as KOH or LiOH in a solvent such as MeOH or

THF. The resulting acid of formula 5.5 is then subjected to an amide bond coupling with an amine of formula 1.8 using HATU or another suitable coupling reagent such as EDCI and HOBT in the presence of a base such as diisopropylethylamine to form the corresponding amide of the formula 5.6.

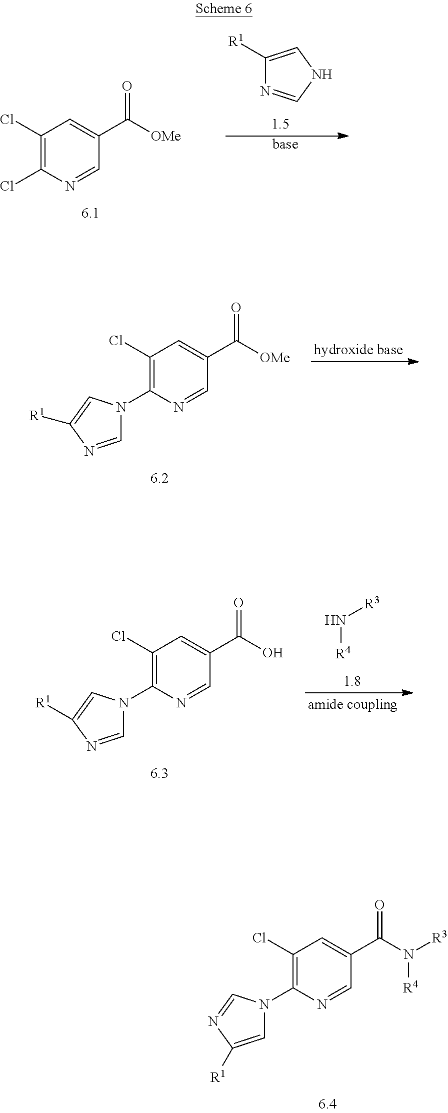

Scheme 6 illustrates a method for the synthesis of compounds of the general formula 6.4. Chloropyridyl derivative 6.1 is coupled with imidazole 1.5 by heating the mixture in the presence of a base such as CsF in a suitable solvent such as DMSO or DMAC. The resulting ester of formula 6.2 is hydrolyzed by treating with aqueous base such as KOH or LiOH in a solvent such as MeOH or THF. The resulting acid of formula 6.3 is then subjected to an amide bond coupling with an amine of formula 1.8 using HATU or another suitable coupling reagent such as EDCI and HOBT in the presence of a base such as diisopropylethylamine in a solvent such as DMSO to form the corresponding amide of the formula 6.4.

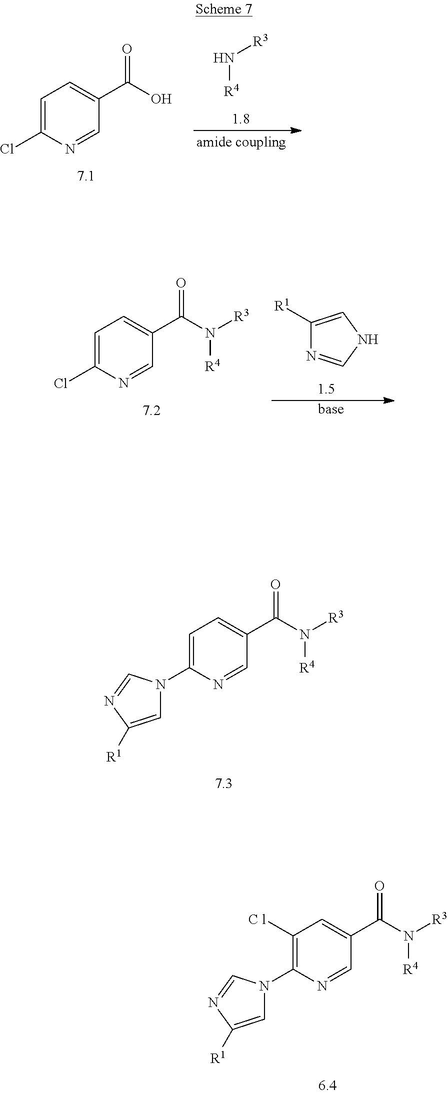

Scheme 7 depicts a method for the synthesis of compounds of the general formula 7.3. 6-Chloronicotinic acid (7.1) is subjected to an amide bond coupling with an amine of formula 1.8 using EDCI and HOBT or another suitable amide bond forming reagent such as HATU in the presence of a base such as diisopropylethylamine in a solvent such as DMSO. The resulting amide of formula 7.2 is then coupled with imidazole 1.5 by heating the mixture in the presence of a base such as CsF in a solvent such as DMSO or DMAC to afford the target compound of formula 7.3.

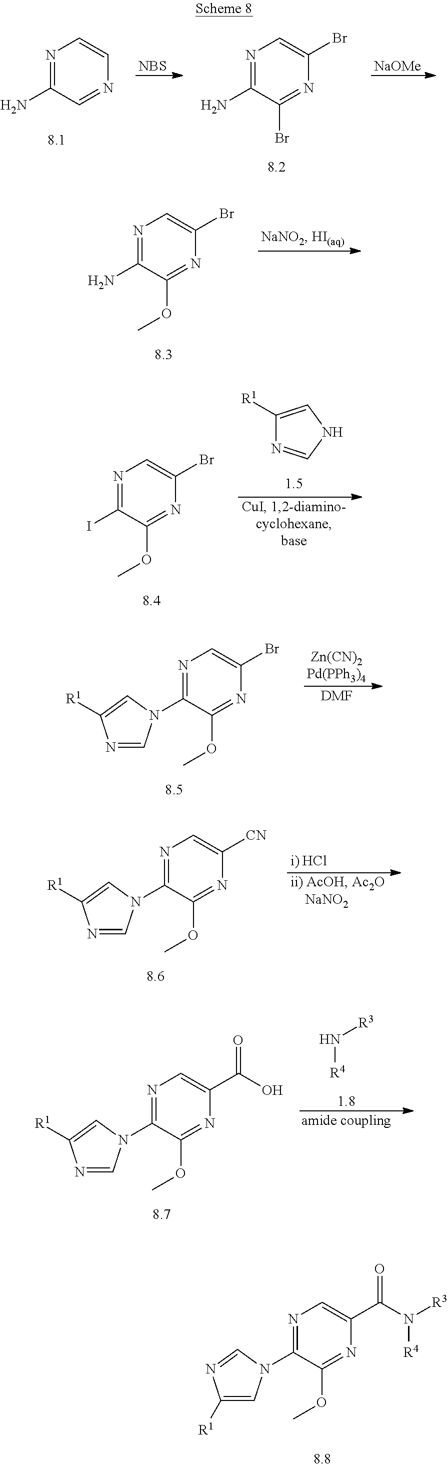

Scheme 8 illustrates a method for preparing amide derivatives of formula 8.8. The synthesis commences with the bromination of 2-aminopyrazine (8.1) using a brominating reagent such as NBS. The resulting dibromopyrazine 8.2 is treated with NaOMe to afford methoxy-substituted intermediate 8.3, which is converted to the corresponding iodide of formula 8.4 by heating in the presence of NaNO₂ and aqueous HI in a solvent such as CH₃CN. The compound of formula 8.4 is then coupled with imidazole 1.5 by heating the mixture in the presence of Cul, 1,2-diaminocyclohexane, and a base such as K₃PO₄ in a solvent such as dioxane. The resulting intermediate of formula 8.5 is converted to the corresponding nitrile of formula 8.6 by heating in the presence of Zn(CN)₂ and Pd(PPh₃)₄ in DMF. The nitrile group of 8.6 is then hydrolyzed to the corresponding acid of formula 8.7 by treating with concentrated HCl followed by AcOH, Ac₂O and NaNO₂. The resulting acid of formula 8.7 is then subjected to an amide bond coupling with an amine of the formula 1.8 using HATU or another suitable coupling reagent such as EDCI and HOBT in the presence of a base such as diisopropylethylamine in a solvent such as DMSO to form the corresponding amide of the formula 8.8.

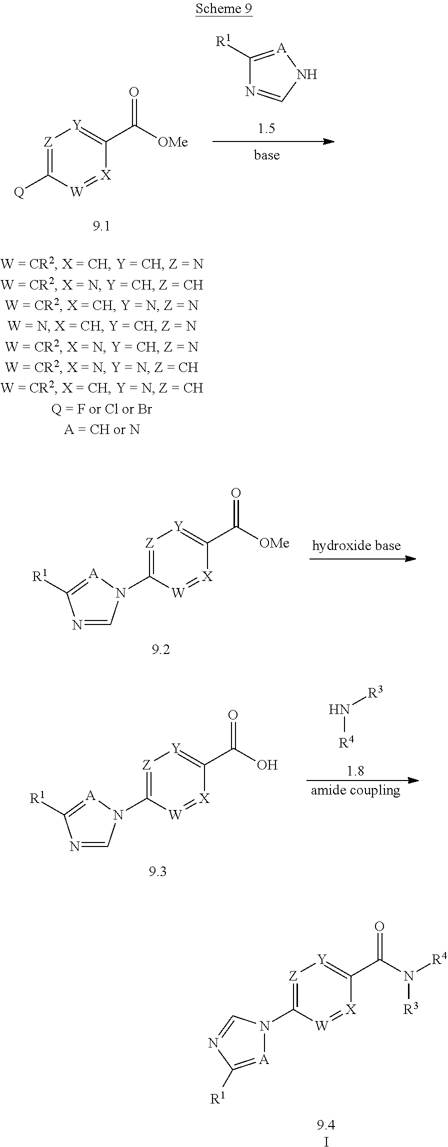

Scheme 9 illustrates a method for the synthesis of compounds of the general formula 9.4. Heteroaryl fluorides 9.1 are coupled with the imidazole or triazole of formula 1.5 by heating the mixture in the presence of a base such as CsF in a suitable solvent such as DMSO or DMAC. This procedure may also be used when Q=Cl and either Z═N (or both W═Z═N). Alternatively, where Q=Br the coupling reaction may be carried out by heating the mixture in the presence of copper iodide and a suitable base such as cesium carbonate in a solvent such as

DMF. The resulting ester of formula 9.2 is hydrolyzed by treating with aqueous base such as KOH or LiOH in a solvent such as MeOH or THF to give the acid of formula 9.3. This material is then subjected to an amide bond coupling with an amine of formula 1.8 using HATU or another suitable coupling reagent such as EDCI and HOBT in the presence of a base such as diisopropylethylamine in a solvent such as DMSO to form the corresponding amide of the formula 9.4.

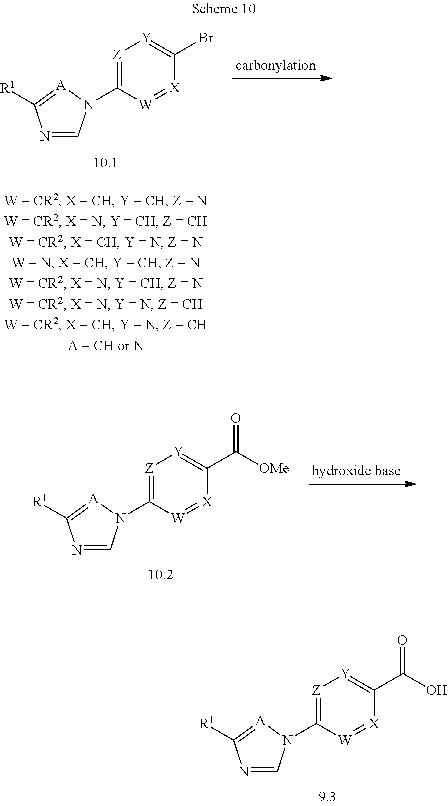

Scheme 10 illustrates an alternative method for the preparation of intermediate 9.3 starting from bromide 10.1 (in the case where R¹═CH₃, W═COCH₃, X═N, Y═Z═CH, A═CH, 10.1 can be made using the method of US 2009062529). A solution of 10.1 and a suitable base such as triethylamine, in a solvent such as MeOH, is heated in the presence of carbon monoxide and a palladium catalyst such as Pd(dppf)₂Cl₂.DCM. The resultant methyl ester 10.2 is then hydrolyzed by exposure to an aqueous solution of a hydroxide base such as sodium, lithium, or potassium hydroxide to afford acid 9.3.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluants/gradients were chosen to provide appropriate R_{f}s or retention times.

### Preparations

### Preparation 1: 4-Methoxy-5-(4-methyl-1H-imidazol-1-yl)pyridine-2-carboxylic acid (P1)

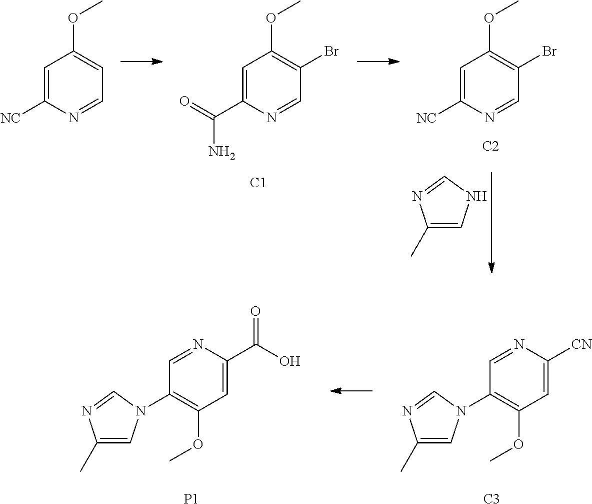

Step 1. Preparation of 5-bromo-4-methoxypyridine-2-carboxamide (C1). N-Bromosuccinimide (69 g, 0.39 mol) was added to a 0° C. solution of 4-methoxypyridine-2-carbonitrile (prepared according to the method of R. T. Shuman et al., J. Org. Chem. 1990, 55, 738-741) (40 g, 0.30 mol) in concentrated sulfuric acid (150 mL). The reaction mixture was heated at 55° C. for 18 hours, then combined with an identical reaction and poured into ice water. After basification to pH 10 with aqueous 8 N sodium hydroxide solution, the mixture was filtered to provide the title product as a yellow solid. Yield: 120 g, 0.519 mol, 86%.

Step 2. Preparation of 5-bromo-4-methoxypyridine-2-carbonitrile (C2). Oxalyl chloride (66 mL, 0.76 mol) was added cautiously in a drop-wise manner to N,N-dimethylformamide (800 mL) at 0° C. Pyridine (106 mL, 1.3 mol) was then added to the ice-cooled mixture, followed after 10 minutes by 5-bromo-4-methoxypyridine-2-carboxamide (C1) from the previous step (60 g, 0.26 mol) in one portion. The reaction mixture was allowed to stir at 0° C. for 1 hour, then was combined with an identical reaction mixture and partitioned between water (500 mL) and ethyl acetate (500 mL). The aqueous layer was extracted with ethyl acetate (2×500 mL), and the combined organic layers were washed with brine, dried over sodium sulfate, filtered and concentrated in vacuo. The residue was purified by chromatography on silica (Gradient: 1:20 to 1:10 ethyl acetate: petroleum ether) to afford the title product as a white solid. Yield: 60 g, 0.28 mol, 54%. ¹H NMR (400 MHz, CDCl₃) δ 3.95 (s, 3H), 7.12 (s, 1H), 8.58 (s, 1H).

Step 3. Preparation of 4-methoxy-5-(4-methyl-1H-imidazol-1-yl)pyridine-2-carbonitrile (C3). 4-Methyl-1H-imidazole (15 g, 0.18 mol), potassium carbonate (34 g, 0.25 mol) and 18-crown-6 (64 g, 0.24 mol) were combined in acetonitrile (600 mL), and the reaction mixture was heated to 60° C. for 2 hours. 5-Bromo-4-methoxypyridine-2-carbonitrile (C2) (25 g, 0.12 mol) was added in one portion, and the reaction was heated to reflux for 18 hours. After being combined with an identical reaction mixture, the reaction was partitioned between water (500 mL) and ethyl acetate (500 mL). The aqueous layer was extracted with ethyl acetate (2×300 mL), and the combined organic layers were washed with brine, dried over sodium sulfate, filtered and concentrated. Purification by chromatography on silica (Gradient: 1:10 to 1:2 ethyl acetate: petroleum ether) provided the title product as a white solid. Yield: 8.2 g, 0.038 mol, 16%. ¹H NMR (400 MHz, CDCl₃) δ 2.30 (d, J=1 Hz, 3H), 4.02 (s, 3H), 6.96-6.97 (m, 1H), 7.38 (s, 1H), 7.82 (d, J=1.5 Hz, 1H), 8.53 (s, 1H).

Step 4. Preparation of 4-methoxy-5-(4-methyl-1H-imidazol-1-yl)pyridine-2-carboxylic acid (P1). A mixture of 4-methoxy-5-(4-methyl-1H-imidazol-1-yl)pyridine-2-carbonitrile (C3) (10.5 g, 49.0 mmol) and potassium hydroxide (5.6 g, 100 mmol) in methanol (100 mL) and water (100 mL) was heated to reflux for 18 hours. The reaction was concentrated in vacuo to remove the majority of the methanol, and the remaining mixture was cooled to 0° C. and acidified to pH 5-6 with concentrated hydrochloric acid. Filtration provided the title product as a white solid. Yield: 9.5 g, 41 mmol, 84%. LCMS m/z 234.0 (M+1). ¹H NMR (400 MHz, DMSO-d₆) δ 2.13 (s, 3H), 3.97 (s, 3H), 7.25 (s, 1H), 7.79 (s, 1H), 7.91 (s, 1H), 8.58 (s, 1H).

### Preparation 2: Lithium 5-chloro-6-(4-methyl-1H-imidazol-1-yl)nicotinate (P2)

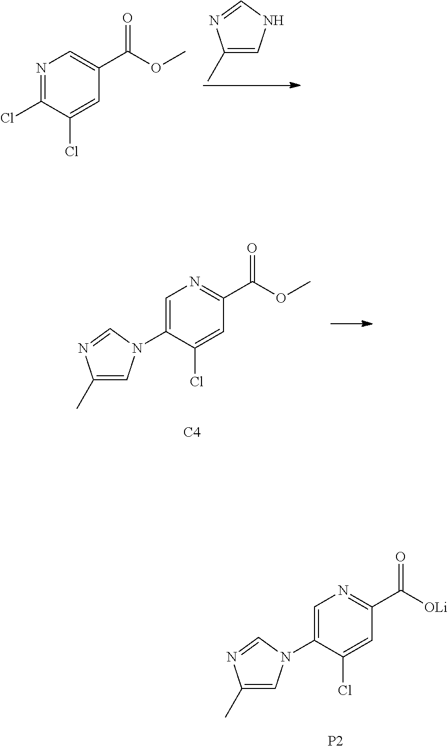

Step 1. Preparation of methyl 5-chloro-6-(4-methyl-1H-imidazol-1-yl)nicotinate (C4). Methyl 5,6-dichloronicotinate (800 mg, 3.88 mmol), 4-methyl-1H-imidazole (638 mg, 7.77 mmol) and cesium fluoride (1.18 g, 7.77 mmol) were combined and the flask was purged with nitrogen. Dimethyl sulfoxide (9.7 mL) was added and the reaction was heated to 100° C. for 15 minutes. The reaction mixture was then partitioned between aqueous sodium bicarbonate solution (100 mL) and ethyl acetate (100 mL), and the aqueous layer was extracted with ethyl acetate (2×50 mL). The combined organic layers were washed with aqueous sodium bicarbonate solution (50 mL) and brine (50 mL), dried over magnesium chloride, filtered and concentrated in vacuo. Chromatography on silica (Gradient: 40% to 80% ethyl acetate in heptane) afforded the title compound as a white solid. Yield: 604 mg, 2.40 mmol, 62%. LCMS m/z 252.4, 254.4 (M+1). ¹H NMR (400 MHz, CDCl₃) δ 2.32 (br s, 3H), 4.00 (s, 3H), 7.48 (m, 1H), 8.32 (d, J=1.2 Hz, 1 H), 8.47 (d, J=1.9 Hz, 1H), 8.98 (d, J=1.9 Hz, 1H).

Step 2. Preparation of lithium 5-chloro-6-(4-methyl-1H-imidazol-1-yl)nicotinate (P2). Aqueous lithium hydroxide solution (2 M, 5.3 mL, 10.6 mmol) was added to a solution of methyl 5-chloro-6-(4-methyl-1H-imidazol-1-yl)nicotinate (C4) (497 mg, 1.97 mmol) in tetrahydrofuran (13.2 mL), and the reaction mixture was stirred at room temperature for 3 hours. The product slowly precipitated out of the reaction to provide a white solid. The supernatant was decanted and the solid was triturated with tetrahydrofuran (5 mL) to provide the title product as a solid. Yield: 609 mg, 2.49 mmol, >100%. LCMS m/z 238.4, 240.4 (M+1). ¹H NMR (400 MHz, DMSO-d₆) δ 2.17 (br s, 3H), 7.39 (m, 1H), 8.06 (d, J=1.2 Hz, 1H), 8.29 (d, J=1.8 Hz, 1H), 8.77 (d, J=1.7 Hz, 1H).

### Preparation 3: 5-Methoxy-6-(4-methyl-1H-imidazol-1-yl)nicotinic acid (P3)

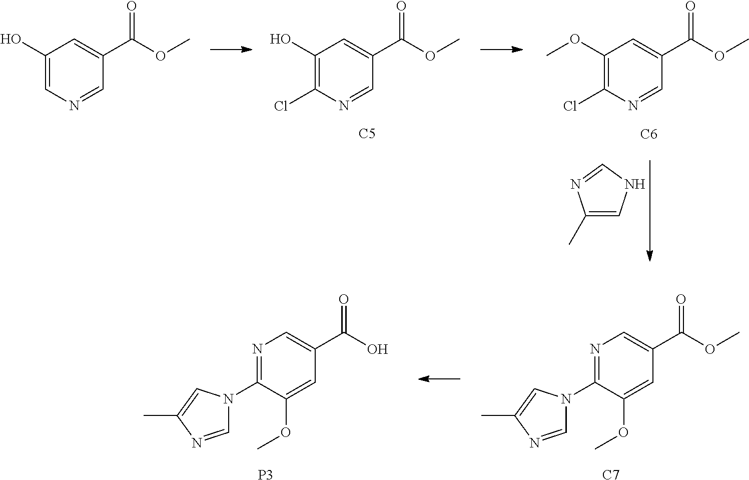

Step 1. Preparation of methyl 6-chloro-5-hydroxynicotinate (C5). N-Chlorosuccinimide (95%, 881 mg, 6.27 mmol) was added to a solution of methyl 5-hydroxynicotinate (800 mg, 5.22 mmol) in N,N-dimethylformamide (5.2 mL). The mixture was stirred at 80° C. for 18 hours, then concentrated in vacuo. Two purifications by chromatography on silica (Gradient: 20% to 100% ethyl acetate in heptane) provided the title product as a white solid. Yield: 306 mg, 1.63 mmol, 31%. LCMS m/z 188.3, 190.3 (M+1). ¹H NMR (400 MHz, CD₃OD) δ 3.92 (s, 3H), 7.77 (d, J=1.9 Hz, 1H), 8.41 (d, J=1.9 Hz, 1H).

Step 2. Preparation of methyl 6-chloro-5-methoxynicotinate (C6). Potassium carbonate (225 mg, 1.63 mmol) was added to a solution of methyl 6-chloro-5-hydroxynicotinate (C5) (306 mg, 1.63 mmol) in acetone (32.6 mL). After addition of methyl iodide (99%, 0.123 mL, 1.96 mmol), the reaction mixture was stirred at 50° C. for 5 hours. Removal of solvent in vacuo was followed by partitioning of the residue between water (50 mL) and ethyl acetate (50 mL). The organic layer was extracted with ethyl acetate (50 mL) and the combined organic layers were washed with brine (30 mL), dried over magnesium sulfate, filtered and concentrated in vacuo. Chromatography on silica (Gradient: 20% to 80% ethyl acetate in heptane) afforded the title compound as a white solid. Yield: 202 mg, 1.00 mmol, 61%. LCMS m/z 202.3, 204.3 (M+1). ¹H NMR (400 MHz, CDCl₃) δ 3.97 (s, 3H), 3.99 (s, 3H), 7.77 (d, J=1.9 Hz, 1H), 8.60 (d, J=1.9 Hz, 1H).

Step 3. Preparation of methyl 5-methoxy-6-(4-methyl-1H-imidazol-1-yl)nicotinate (C7). Methyl 6-chloro-5-methoxynicotinate (C6) (185 mg, 0.918 mmol) was combined with 4-methyl-1H-imidazole (148 mg, 1.80 mmol) and cesium fluoride (273 mg, 1.80 mmol). After the mixture was purged with nitrogen, dimethyl sulfoxide (3.0 mL) was added and the mixture was heated at 110° C. for 1.25 hours. After cooling to room temperature, the reaction was combined with an identical reaction carried out on 0.15 mmol of substrate, and poured into aqueous sodium bicarbonate solution (25 mL). After extraction with ethyl acetate (3×25 mL), the organic layers were combined, washed with aqueous sodium bicarbonate solution (25 mL), washed with brine (25 mL), dried over magnesium sulfate and concentrated in vacuo. Chromatography on silica (Gradient: 0% to 40% [9:1 ethyl acetate: 2 M ammonia in methanol] in ethyl acetate), afforded the title product. Yield: 148 mg, 0.599 mmol, 56%. LCMS m/z 248.5 (M+1). ¹H NMR (400 MHz, CDCl₃) δ 2.30 (d, J=0.9 Hz, 3H), 3.98 (s, 3H), 4.04 (s, 3H), 7.63 (m, 1H), 7.93 (d, J=1.7 Hz, 1H), 8.47 (d, J=1.1 Hz, 1H), 8.68 (d, J=1.8 Hz, 1H).

Step 4. Preparation of 5-methoxy-6-(4-methyl-1H-imidazol-1-yl)nicotinic acid (P3). Methyl 5-methoxy-6-(4-methyl-1H-imidazol-1-yl)nicotinate (C7) (23 mg, 0.093 mmol) was dissolved in tetrahydrofuran (0.93 mL), and aqueous lithium hydroxide solution (2 M, 0.37 mL, 0.74 mmol) was added. The reaction mixture was stirred for 3 hours at room temperature, then acidified with aqueous hydrochloric acid (6 M, 0.5 mL) and concentrated under reduced pressure. The resulting title product was used without further purification. LCMS m/z 234.4 (M+1).

### Preparation 4: 6-Methoxy-5-(4-methyl-1H-imidazol-1-yl)pyridine-2-carboxylic acid lithium salt (P4)

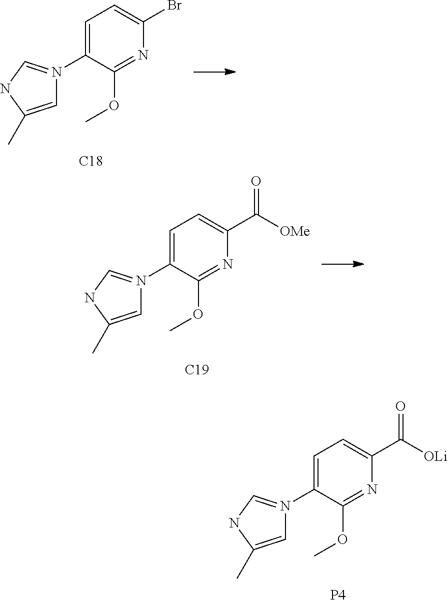

Step 1. Synthesis of methyl 6-methoxy-5-(4-methyl-1H-imidazol-1-yl)pyridine-2-carboxylate (C19). To a solution of the known 6-bromo-2-methoxy-3-(4-methyl-1H-imidazol-1-yl)pyridine (C18, US 2009062529) (44.2 g, 165 mmol) in MeOH (165 mL) was added TEA (46 mL, 330 mmol, 2 eq) and Pd(dppf)₂Cl₂.DCM (6.7 g, 8.24 mmol, 0.05 eq). The mixture was degassed several times with N₂. The reaction was heated to 70° C. under CO atmosphere (3 bar) in a Parr apparatus. After 30 min, the pressure dropped to 0.5 bar. Additional CO was added until the pressure stayed constant for a period of 30 min. The mixture was allowed to cool to RT and filtered through a pad of Celite. The filtrate was washed with MeOH (2×) and concentrated under reduced pressure. The residue (88 g) was dissolved in EtOAc (1 L) and H₂O (700 mL), and the layers were separated. The organic layer was washed with H₂O (200 mL), and the aqueous layer was extracted with EtOAc (500 mL). The combined organic layers were dried over MgSO₄, filtered and concentrated to provide the title compound. Yield: 42.6 g, 175 mmol, quant.

Step 2. Synthesis of 6-methoxy-5-(4-methyl-1H-imidazol-1-yl)pyridine-2-carboxylic acid lithium salt (P4). To a solution of methyl 6-methoxy-5-(4-methyl-1H-imidazol-1-yl)pyridine-2-carboxylate (C19) (41.6 g, 168 mmol) in MeOH (832 mL) at 0° C. was added drop-wise a solution of lithium hydroxide (4.23 g, 177 mmol, 1.05 eq) in H₂O (277 mL). The mixture was stirred at RT for 16 h, whereupon it was concentrated under reduced pressure. The residue was co-evaporated from toluene (2×) and the resulting solid was dried in a vacuum oven at 45° C. for 16 h to afford the title compound as a brown solid. Yield: 47.24 g, 198 mmol, assumed quantitative. LCMS m/z 234.1 (M+1). ¹H NMR (400 MHz, DMSO-d₆) δ 2.11 (s, 3H), 3.94 (s, 3H), 7.18 (s, 1H), 7.59 (d, J=7.6 Hz, 1H), 7.78 (d, J=7.6 Hz, 1H), 7.84 (s, 1H).

### Preparation 5: 5-Chloro-4-fluoro-2,3-dihydro-1-benzofuran-3-amine (P5)

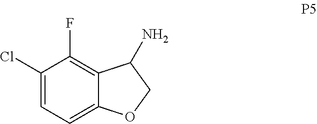

Step 1. Synthesis of 5-chloro-4-fluoro-2,3-dihydro-1-benzofuran-3-ol. Trimethylsulfoxonium iodide (158 g, 716 mmol) was added to a solution of 3-chloro-2-fluoro-6-hydroxybenzaldehyde (50.0 g, 290 mmol) in DMSO (955 mL) followed by KOt-Bu (80.4 g, 716 mmol). After TLC indicated starting material consumption, the reaction mixture was poured into ice water. The reaction mixture was extracted with EtOAc (3×). The organic layers were combined, diluted with heptanes, and washed with water (3×). The organic solution was dried over Na₂SO₄, and concentrated under reduced pressure to afford an orange solid. The residue was slurried in heptane, and the desired product was isolated by filtration to provide the title compound as a solid. Yield: 32.4 g, 60%. ¹H NMR (400 MHz, CDCl₃) δ 4.50 (dd, J=10.7, 2.5 Hz, 1H), 4.59 (dd, J=10.7, 6.4 Hz, 1H), 5.58 (dd, J=6.6, 2.5 Hz, 1H), 6.61 (d, J=8.6 Hz, 1H), 7.24 (m, 1H).

Step 2. Synthesis of 3-azido-5-chloro-4-fluoro-2,3-dihydro-1-benzofuran. Diphenylphosphoryl azide (28.4 mL, 127 mmol) was added to a 0° C. solution of 5-chloro-4-fluoro-2,3-dihydro-1-benzofuran-3-ol (20 g, 110 mmol) in toluene (220 mL), followed by DBU (17.8 mL, 127 mmol). The reaction mixture was stirred at 0° C. for 1 h and 45 min, whereupon it was allowed to warm to room temperature and stirred for an additional 2 h. The reaction was washed with water (2×) and a 5% aqueous solution of HCl. The organic layer was dried over Na₂SO₄ and concentrated under reduced pressure. Chromatography (Gradient: 100% heptane to 5% EtOAc in heptane) provided the title compound as a clear oil which solidified upon standing. Yield: 11.8 g, 51%. ¹H NMR (400 MHz, CDCl₃) δ 4.54 (dd, J=10.5, 2.7 Hz, 1H), 4.65 (dd, J=10.7, 7.4 Hz, 1H), 5.31 (dd, J=7.2, 2.5 Hz, 1H), 6.67 (d, J=8.6 Hz, 1H), 7.32 (dd, J=8.6, 7.6 Hz, 1H).

Step 3. Synthesis of 5-chloro-4-fluoro-2,3-dihydro-1-benzofuran-3-amine (P5). Polymer-supported triphenylphosphine (100 g, 1.48 mmol/g loading) was added to a solution of 3-azido-5-chloro-4-fluoro-2,3-dihydrobenzofuran (11.8 g, 55.3 mmol) in heptane (5.76 mL), THF (1.3 L), and water (30.0 mL) in a 3-neck flask equipped with a mechanical stirrer. The reaction was heated to 50° C., and after 10 min an exotherm was noted (internal reaction temperature of 57° C.). The reaction was cooled to room temperature and was stirred for an additional 2 h. Et₂O (500 mL) was added to the reaction, followed by MgSO₄ (250 g), and the reaction was stirred at room temperature for 0.5 h. The reaction mixture was filtered through Celite, and the solids were washed with Et₂O. The filtrate was concentrated to a minimal volume, and aqueous 4 M HCl (20 mL) was added. The resulting precipitate was isolated by filtration and dried under reduced pressure to afford the title compound as its HCl salt (11.1 g, 90%). ¹H NMR (400 MHz, DMSO-d₆) δ 4.80-4.71 (m, 2H), 5.26 (dd, J=7.2, 2.9 Hz, 1H), 6.87 (d, J=8.6 Hz, 1H), 7.56 (dd, J=8.6, 8.2 Hz, 1H), 8.90 (br s, 3H). This material was taken up in CH₂Cl₂ and washed once with aqueous 1 M NaOH. The aqueous layer was extracted twice with CH₂Cl₂. The combined organic layers were dried (Na₂SO₄), filtered, and concentrated under reduced pressure to provide title compound as the free amine. This material was used without further purification.

### Preparation 6: 5-Chloro-2,3-dihydro-1-benzofuran-3-amine (P6) enantiomer 1 and 2

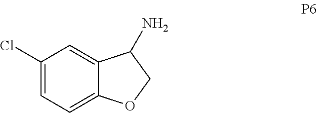

Step 1. Synthesis of methyl 5-chloro-2-hydroxybenzoate. Concentrated sulfuric acid (20 mL) was added to a suspension of 5-chlorosalicylic acid (50 g, 290 mmol) in methanol (500 mL), and the mixture was refluxed for five days. The reaction was concentrated under reduced pressure and the residue was dissolved in Et₂O (500 mL). The resulting mixture was poured into a saturated aqueous solution of NaHCO₃ (400 mL) cooled to 0° C., and the layers were separated. The aqueous layer was extracted with Et₂O (2×400 mL) and the combined organic layers were washed with a saturated aqueous solution of NaHCO₃ and brine. The organic layer was dried (Na₂SO₄) and concentrated under reduced pressure to afford the title compound as a white solid. Yield: 49.5 g, 265 mmol, 91%.

Step 2. Synthesis of methyl 5-chloro-2-(2-ethoxy-2-oxoethoxy)benzoate. Ethyl bromoacetate (30 mL, 265 mmol) was added to a suspension of methyl 5-chloro-2-hydroxybenzoate (49.5 g, 265 mmol) and K₂CO₃ (128 g, 929 mmol) in acetone (1.0 L). The mixture was refluxed overnight, whereupon the reaction was allowed to cool to room temperature. After filtration, the filtrate was concentrated under reduced pressure and the residue was dissolved in CH₂Cl₂. The resulting solution was washed twice with water, dried (Na₂SO₄), and concentrated under reduced pressure to afford the title compound as a red wax. Yield: 55 g, 202 mmol, 76%.

Step 3. Synthesis of 5-chloro-1-benzofuran-3(2H)-one. KOt-Bu (48.1 g, 429 mmol) was added in portions to a solution of methyl 5-chloro-2-(2-ethoxy-2-oxoethoxy)benzoate (46.8 g, 171 mmol) in THF (2 L) at 0° C. The mixture was stirred at 0° C. for 2 h, whereupon a saturated aqueous solution of NH₄Cl (500 mL) was added followed by EtOAc (500 mL). The layers were separated and the aqueous layer was extracted with EtOAc (2×1 L). The combined organic layers were washed with brine, dried over Na₂SO₄, and concentrated under reduced pressure to give a mixture of the title compound and ethyl 5-chloro-3-hydroxy-1-benzofuran-2-carboxylate. This mixture was dissolved in DMSO (260 mL) and water (450 mL) and LiOH.H₂O (33.0 g, 803 mmol) was added. The reaction was stirred at 70° C. for 3 hours and then at room temperature overnight. The mixture was poured into a 10% aqueous solution of HCl (1 L) resulting in the formation of a solid precipitate, which was collected by filtration and washed with water. The solid material was dissolved in Et₂O and washed with water. The combined organic layers were washed with brine, dried over Na₂SO₄, and concentrated under reduced pressure. Chromatography on silica (Gradient: 0% to 40% ethyl acetate in heptane) provided the title compound as a red solid. Yield: 19.6 g, 117 mmol, 68% over 2 steps.

Step 4. Synthesis of 5-chloro-2,3-dihydro-1-benzofuran-3-ol. NaBH₄ (1.68 g, 44 mmol) was added to a solution of 5-chloro-1-benzofuran-3(2H)-one (9.93 g, 59.1 mmol) in MeOH (600 mL) at 0° C. The mixture was stirred at 0° C. for 2 h and at room temperature for 2 h, whereupon water (500 mL) was added. The reaction mixture was concentrated under reduced pressure to remove most of the MeOH. EtOAc (800 mL) was added and the layers were separated. The aqueous layer was extracted with EtOAc (800 mL), and the combined organic layers were washed with brine, dried over Na₂SO₄, and concentrated under reduced pressure to give the title compound as a red wax. Yield: 9.75 g, 57 mmol, 97%.

Step 5. Synthesis of 3-azido-5-chloro-2,3-dihydro-1-benzofuran. To a solution of 5-chloro-2,3-dihydro-1-benzofuran-3-ol (9.75 g, 57 mmol) in toluene (200 mL) at 0° C. was added 1,8-diazabicyclo[5.4.0]undec-7-ene (DBU) (10.2 mL, 68.4 mmol) followed by diphenylphosphoryl azide (DPPA) (14.8 mL, 68.4 mmol). The mixture was stirred at 0° C. for 3 h and then at room temperature overnight. ‘H NMR indicated 85% conversion of the starting material. The mixture was cooled to 0° C. and additional DBU (2.56 mL, 17.1 mmol) was added followed by DPPA (3.7 mL, 17.1 mmol). The reaction was stirred at 0° C. for 1 h, whereupon ¹H NMR showed that the reaction had reached completion. Water (90 mL) was added to the reaction mixture followed by an aqueous solution of HCl (1 N, 90 mL). The layers were separated and the aqueous layer was extracted three times with CH₂Cl₂. The combined organic layers were dried over Na₂SO₄ and concentrated under reduced pressure. Chromatography on silica (Gradient: 0% to 10% EtOAc in heptane) provided the title compound as a yellow oil. Yield: 6.1 g, 31.3 mmol, 55%.

Step 6. Synthesis of 5-chloro-2,3-dihydro-1-benzofuran-3-amine hydrochoride salt. To a solution of 3-azido-5-chloro-2,3-dihydrobenzofuran (6.10 g, 31.3 mmol) in THF (260 mL) were sequentially added water (5.63 mL) and triphenylphosphine (24.7 g, 94 mmol). The reaction was stirred at 50° C. overnight, whereupon it was allowed to cool to room temperature and diluted with Et₂O (500 mL). HCl in dioxane (4 N, 8.25 mL, 33 mmol) was added and the solution was stirred for 5 min at room temperature, whereupon the precipitate was collected by filtration to afford the title compound as a white solid. Yield: 5.9 g, 28.9 mmol, 92%.

Step 7. Synthesis of 5-chloro-2,3-dihydro-1-benzofuran-3-amine. 5-Chloro-2,3-dihydro-1-benzofuran-3-amine hydrochoride salt (10.8 g, 53 mmol) was dissolved in saturated aqueous NaHCO₃ solution (300 mL). The pH was adjusted to 9 by the addition of aqueous NaOH solution (3 N), and the mixture was extracted with CH₂Cl₂/MeOH (90/10) and CHCl₃/MeOH (90/10). The combined organic layers were dried over MgSO₄ and concentrated under reduced pressure to give the title compound. Yield: 5.00 g, 29.6 mmol, 56%. ¹H NMR of the aqueous layer indicated the presence of additional product. The aqueous layer was concentrated to dryness and the residue was stirred in CHCl₃/MeOH (80/20) overnight. The mixture was filtered and the filtrate was concentrated under reduced pressure to furnish additional title compound (0.50 g, 2.96 mmol, 5%). ¹H NMR of the MgSO₄ pad indicated the presence of a significant amount of the desired product. The solids were suspended in a mixture of isopropanol (420 mL) and a 7 N solution of ammonia in MeOH (7 mL) and stirred for 15 minutes. The solids were removed by filtration and the filtrate was concentrated under reduced pressure to afford an additional 3.24 g (19.2 mmol, 36%) of the title compound. The title compound was obtained as a white solid. Combined yield: 8.74 g, 51.7 mmol, 98%.

Step 8. Synthesis of 5-chloro-2,3-dihydro-1-benzofuran-3-amine enantiomer 1 (P6 entantiomer 1). Racemic 5-chloro-2,3-dihydro-1-benzofuran-3-amine (8.74 g, 51.7 mmol) and (+)-phencyphos (2-hydroxy-5,5-dimethyl-4-phenyl-1,3,2-dioxaphosphorinan-2-one) (12.52 g, 51.7 mmol) were suspended in EtOH (300 mL) and water (2 mL). The mixture was heated to reflux using a heat gun and then allowed to cool slowly to room temperature overnight. The resulting solid was isolated by filtration and recrystallized from EtOH/water (120 mL/0.7 mL). The solids were dissolved in aqueous NaOH (3 N, 70 mL) and CH₂Cl₂ (100 mL) and stirred at room temperature for 2 h, whereupon the mixture was filtered to remove the (+)-phencyphos sodium salt. The solids were washed with CH₂Cl₂ and the two layers from the combined filtrate and washings were separated. The aqueous layer was extracted with CH₂Cl₂ and the combined organic layers were dried by adding Na₂SO₄ and stirring for 10 min followed by filtration through a pad of Na₂SO₄ to afford the title compound as a yellow oil. Yield: 2.92 g, 17.3 mmol, 33%, 96% ee. The spectral data was identical to that of enantiomer 2 in Step 9.

Step 9. Synthesis of 5-chloro-2,3-dihydro-1-benzofuran-3-amine enantiomer 2 (P6 enantiomer 2). The mother liquor from the first filtration in step 8 was concentrated under reduced pressure to afford 11.65 g of the (+)-phencyphos salt of 5-chloro-2,3-dihydro-1-benzofuran-3-amine enantiomer 2 (28.3 mmol, 59% ee). The solid was dissolved in a mixture of sec-butanol (200 mL) and aqueous KOH solution (1 M, 100 mL) and the layers were separated. To the organic layer (containing the 5-chloro-2,3-dihydro-1-benzofuran-3-amine enantiomer 2 free base, 59% ee) was added (−)-phencyphos (6.78 g, 28 mmol) and the mixture was concentrated under reduced pressure. To the solid was added EtOH (150 mL) and the mixture was heated to reflux using a heat gun and then allowed to cool slowly to room temperature overnight. The resulting solid was isolated by filtration (42% ee) and the filtrate was concentrated under reduced pressure to afford the (−)-phencyphos salt of 5-chloro-2,3-dihydro-1-benzofuran-3-amine enantiomer 2 (7.16 g, 17.3 mmol, 80% ee). Two recrystallizations from EtOH increased the ee to 85%. The resulting solid was dissolved in aqueous NaOH solution (3 N, 60 mL) and CH₂Cl₂ (100 mL) and the mixture was stirred at room temperature for 2 h, whereupon it was filtered. The solids were rinsed with CH₂Cl₂, and the two layers from the filtrate and washings were separated. The aqueous layer was further extracted with CH₂Cl₂ and the combined organic layers were dried by adding Na₂SO₄ and stirring for 10 min followed by filtration through a pad of Na₂SO₄ to remove the remaining (−)-phencyphos sodium salt. The filtrate was concentrated under reduced pressure to afford the title compound. Yield: 1.47 g, 8.7 mmol, 85% ee. To this material was added (−)-phencyphos (2.1 g, 8.7 mmol), EtOH (40 mL) and water (0.1 mL). The mixture was heated to reflux by using a heat gun and then allowed to cool slowly to room temperature overnight. The resulting solid was isolated by filtration (97% ee). Another batch of 5-chloro-2,3-dihydro-1-benzofuran-3-amine enantiomer 2 (42% ee, 2.72 g, 16 mmol, from the first recrystallization in step 9) and (−)-phencyphos (3.87 g, 16 mmol) were suspended in EtOH (70 mL) and water (0.3 mL). The mixture was heated to reflux using a heat gun and then allowed to cool slowly to room temperature overnight. The resulting solid was isolated by filtration (93% ee) and recrystallized from EtOH/water (35 mL/0.5 mL) and again isolated by filtration (97% ee). The two batches of the 5-chloro-2,3-dihydro-1-benzofuran-3-amine enantiomer 2 (−)-phencyphos salt were combined (97% ee, 7.3 g, 17.8 mmol) and dissolved in NaOH solution (3 N, 80 mL) and CH₂Cl₂ (100 mL). The mixture was stirred at room temperature for 2 h, whereupon it was filtered to remove the (−)-phencyphos sodium salt. The solids were rinsed with CH₂Cl₂, and the two layers from the filtrate and washings were separated. The aqueous layer was further extracted with CH₂Cl₂ and the combined organic layers were dried by adding Na₂SO₄ and stirring for 10 min followed by filtration through a pad of Na₂SO₄ to remove the remaining (−)-phencyphos sodium salt. The filtrate was concentrated under reduced pressure to afford the title compound as a pale yellow oil. Yield: 3 g, 17.8 mmol, 34%, ee >97%. The absolute configuration was not determined. LCMS m/z 153.0 [(M-NH₃)+1]. ¹H NMR (400 MHz, CDCl₃) δ 4.16 (dd, J=9.2, 4.7 Hz, 1H), 4.56-4.68 (m, 2H), 6.71 (d, J=8.6 Hz, 1H), 7.11 (dd, J=8.4, 2.2 Hz, 1H), 7.24 (br s, 1H).

### Preparation 7: 5-(Trifluoromethyl)-2,3-dihydro-1-benzofuran-3-amine (P7) (racemic)

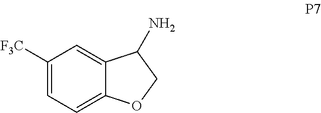

Step 1. Synthesis of N-{(1E)-[2-hydroxy-5-(trifluoromethyl)phenyl]methylene}-2-methylpropane-2-sulfinamide. Cs₂CO₃ (6.23 g, 19.1 mmol) was added to a solution of 2-hydroxy-5-(trifluoromethyl)benzaldehyde (1.65 g, 8.68 mmol) and tert-butylsulfinamide (2.17 g, 17.4 mmol) in CH₂Cl₂ (87 mL). The reaction mixture was heated to reflux overnight and was then allowed to cool to room temperature. The mixture was filtered through Celite, and the filtrate was concentrated under reduced pressure. Chromatography on silica (Gradient: 10% to 80% EtOAc in heptane) furnished the title compound as a white solid. Yield: 1.59 g, 5.42 mmol, 62%. LCMS m/z 294.2 (M+1). ¹H NMR (400 MHz, CDCl₃) δ 1.25 (s, 9H), 7.10 (d, J=8.8 Hz, 1H), 7.65 (dd, J=8.6, 2.2 Hz, 1H), 7.75 (d, J=2.0 Hz, 1H), 8.71 (s, 1H), 11.43 (s, 1H).

Step 2. Synthesis of 2-methyl-N-[5-(trifluoromethyl)-2,3-dihydro-1-benzofuran-3-yl]propane-2-sulfinamide. KOt-Bu (608 mg, 5.42 mmol) was added to a solution of N-{(1E)-[2-hydroxy-5-(trifluoromethyl)phenyl]methylene}-2-methylpropane-2-sulfinamide (1.59 g, 5.42 mmol) and trimethylsulfoxonium iodide (1.19 g, 5.42 mmol) in DMSO (27 mL). The reaction was stirred at room temperature overnight whereupon it was poured into water cooled to 0° C. The mixture was extracted three times with EtOAc and the combined organic layers were dried over MgSO₄ and concentrated under reduced pressure. Chromatography on silica (Gradient: 30% to 80% EtOAc in heptane) afforded the title compound. Yield: 100 mg, 0.33 mmol, 6%. LCMS m/z 308.1 (M+1). ¹H NMR (400 MHz, CDCl₃, mixture of diastereomers) δ 1.21 (s, 3.6H), 1.23 (s, 5.4H), 3.46-3.60 (m, 1H), 4.46 (dd, J=10.2, 4.5 Hz, 0.4H), 4.59 (dd, J=10.5, 4.9 Hz, 0.6H), 4.72 (dd, J=10.2, 8.2 Hz, 0.4H), 4.80 (dd, J=10.5, 8.2 Hz, 0.6H), 5.07-5.22 (comp, 1H), 6.88 (d, J=8.8 Hz, 0.4H), 6.90 (d, J=8.6 Hz, 0.6H), 7.47-7.52 (comp, 1H), 7.54 (s, 0.6H), 7.75 (s, 0.4H).

Step 3. Synthesis of 5-(trifluoromethyl)-2,3-dihydro-1-benzofuran-3-amine (P7). 4 M HCl in dioxane (0.24 mL, 0.96 mmol) was added to a solution of 2-methyl-N-[5-(trifluoromethyl)-2,3-dihydro-1-benzofuran-3-yl]propane-2-sulfinamide (100 mg, 0.33 mmol) in MeOH (2.5 mL). The reaction was stirred at room temperature for 1.5 h, whereupon the reaction was concentrated under reduced pressure to afford the crude title compound as a white solid. GCMS m/z 187 (M−NH₂). The crude material was used directly in the ensuing amide coupling reaction without further purification.

### EXAMPLES

### Example 1

### 6-(4-Methyl-1H-imidazol-1-yl)-N-({4-[3-(trifluoromethyl)phenyl]tetrahydro-2H-pyran-4-yl}methyl)nicotinamide (1)

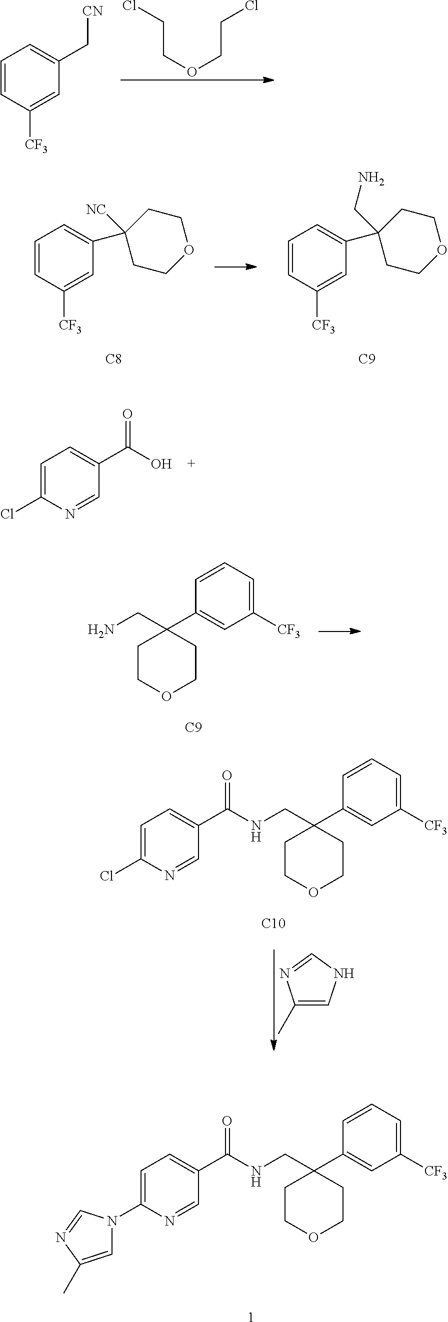

Step 1. Synthesis of 1-{443-(trifluoromethyl)phenyl]tetrahydro-2H-pyran-4-yl}methanamine (C9).

A. Synthesis of 4-[3-(trifluoromethyl)phenyl]tetrahydro-2H-pyran-4-carbonitrile (C8). [3-(Trifluoromethyl)phenyl]acetonitrile (40.7 g, 220 mmol) and bis(2-chloroethyl) ether (25.8 mL, 220 mmol) were dissolved in N,N-dimethylformamide (800 mL). Sodium hydride (60% suspension in mineral oil, 17.6 g, 440 mmol) was added in small portions over 1.5 hours, such that the temperature of the reaction did not exceed 50-55° C. After completion of the addition, the reaction mixture was stirred at 55° C. for 2 hours, and then stirred at room temperature for 18 hours. Excess sodium hydride was slowly decomposed by drop-wise addition of water until hydrogen evolution ceased. The mixture was diluted with water (2 L), and extracted with ethyl acetate (3×300 mL). The combined extracts were washed with brine, dried over sodium sulfate, and concentrated in vacuo. Chromatography on silica (Eluant: carbon tetrachloride, then 85:15 carbon tetrachloride: ethyl acetate) provided the title compound. Yield: 48 g, 188 mmol, 85%. ¹H NMR (400 MHz, DMSO-d₆) δ 2.08-2.19 (m, 4H), 3.68 (ddd, J=11.5, 11.5, 2.9 Hz, 2H), 4.03 (m, 2H), 7.71 (dd, J=7.8, 7.8 Hz, 1H), 7.77 (br d, J=7.8 Hz, 1H), 7.86 (br s, 1 H), 7.90 (br d, J=7.8 Hz, 1 H).

B. Synthesis of 1-{4-[3-(trifluoromethyl)phenyl]tetrahydro-2H-pyran-4-yl)methanamine (C9). A solution of 4-[3-(trifluoromethyl)phenyl]tetrahydro-2H-pyran-4-carbonitrile (C8) (55.9 g, 219 mmol) in an ammonia/ methanol mixture (825 mL) was purged with argon, and Raney Nickel (30 g) was added. The reaction mixture was purged with hydrogen and stirred under a hydrogen balloon at room temperature, until the reaction was complete as monitored by thin layer chromatography (about 24 hours). The reaction mixture was filtered through Celite, and the filtrate was concentrated in vacuo. Chromatography on silica (Gradient: 0% to 5% methanol in [chloroform containing 1% diethylamine]) afforded the title compound. Yield: 46.3 g, 179 mmol, 82%. LCMS m/z 260.1 (M+1). ¹H NMR (400 MHz, DMSO-d₆) δ 1.86 (ddd, J=13.7, 8.8, 3.9 Hz, 2H), 2.02 (m, 2H), 2.69 (s, 2H), 3.36 (ddd, J=11.3, 8.6, 2.9 Hz, 2H), 3.68 (ddd, J=11.5, 6.4, 3.9 Hz, 2H), 7.58 (m, 3H), 7.65 (m, 1H).

Step 2. Synthesis of 6-chloro-N-({4-[3-(trifluoromethyl) phenyl]tetrahydro-2H-pyran-4-yl)methyl)nicotinamide (C10). 1-{443-(Trifluoromethyl)phenyl]tetrahydro-2H-pyran-4-yl)methanamine (C9) (1.65 g, 6.36 mmol) was combined with 6-chloronicotinic acid (1.00 g, 6.35 mmol),1H-benzotriazol-1-ol (1.03 g, 7.62 mmol) and diisopropylethylamine (4.42 mL, 25.4 mmol) in N,N-dimethylformamide (25 mL), and the mixture was stirred until dissolution was complete. N-[3-(Dimethylamino)propyl]-N′-ethylcarbodiimide hydrochloride (1.46 g, 7.62 mmol) was added, and the reaction was stirred at room temperature for 18 hours, then poured into aqueous sodium bicarbonate solution (150 mL) and extracted with ethyl acetate (3×100 mL). The combined organic layers were washed with saturated aqueous sodium bicarbonate solution (60 mL) and brine (60 mL), and dried over magnesium sulfate. Filtration and removal of solvents under reduced pressure provided a residue, which was chromatographed on silica (Gradient: 30% to 70% ethyl acetate in heptane) to afford the title compound as a solid. Yield: 1.85 g, 4.64 mmol, 73%. LCMS m/z 399.1 (M+1). ¹H NMR (400 MHz, CDCl₃) δ 2.02 (ddd, J=13.9, 7.8, 3.5 Hz, 2H), 2.16 (br ddd, J=14, 7, 3 Hz, 2H), 3.64 (ddd, J=11.9, 7.6, 3.3 Hz, 2H), 3.74 (d, J=6.4 Hz, 2H), 3.91 (ddd, J=11.9, 6.8, 3.5 Hz, 2H), 5.65 (br t, J=6 Hz, 1 H), 7.38 (dd, J=8.3, 0.7 Hz, 1H), 7.56-7.62 (m, 4H), 7.90 (dd, J=8.4, 2.5 Hz, 1H), 8.50 (dd, J=2.5, 0.8 Hz, 1H).

Step 3. Synthesis of 6-(4-methyl-1H-imidazol-1-yl)-N-({4- [3-(trifluoromethyl)phenyl]tetrahydro-2H-pyran-4-yl}methyl)nicotinamide (1). 6-Chloro-N-({4-[3-(trifluoromethyl)phenyl]tetrahydro-2H-pyran-4-yl}methyl)nicotinamide (C10) (150 mg, 0.376 mmol) was combined with 4-methyl-1H-imidazole (61.7 mg, 0.751 mmol) and cesium fluoride (114 mg, 0.750 mmol). After the mixture was purged with nitrogen, dimethyl sulfoxide (0.94 mL) was added and the mixture was heated at 140° C. for 18 hours. After cooling to room temperature, the reaction was poured into water (50 mL) and aqueous sodium bicarbonate solution (20 mL) and then extracted with ethyl acetate (3×50 mL). The combined organic layers were washed with brine (50 mL), dried over magnesium sulfate, filtered and concentrated in vacuo. Chromatography on silica (Gradient: 30% to 100% [9:1 ethyl acetate: 2% ammonia in methanol] in ethyl acetate), afforded the title product as a glass. Yield: 82 mg, 0.18 mmol, 49%. LCMS m/z 445.2 (M+1). ¹H NMR (400 MHz, CDCl₃) δ 2.04 (ddd, J=14.0, 7.8, 3.7 Hz, 2H), 2.17 (br ddd, J=14, 7, 3 Hz, 2H), 2.27 (s, 3H), 3.64 (ddd, J=11.8, 7.6, 3.2 Hz, 2H), 3.75 (d, J=6.4 Hz, 2H), 3.91 (ddd, J=11.9, 6.7, 3.4 Hz, 2H), 5.93 (br s, 1H), 7.29 (br d, J=8.6 Hz, 1H), 7.32 (br s, 1H), 7.57-7.61 (m, 4H), 8.05 (dd, J=8.5, 2.3 Hz, 1H), 8.24 (br s, 1H), 8.58 (br d, J=2.3 Hz, 1H).

### Example 2

### 6-Methoxy-5-(4-methyl-1H-imidazol-1-yl)-N-[(1-phenylcyclopentyl)methyl]pyrazine-2-carboxamide (2)

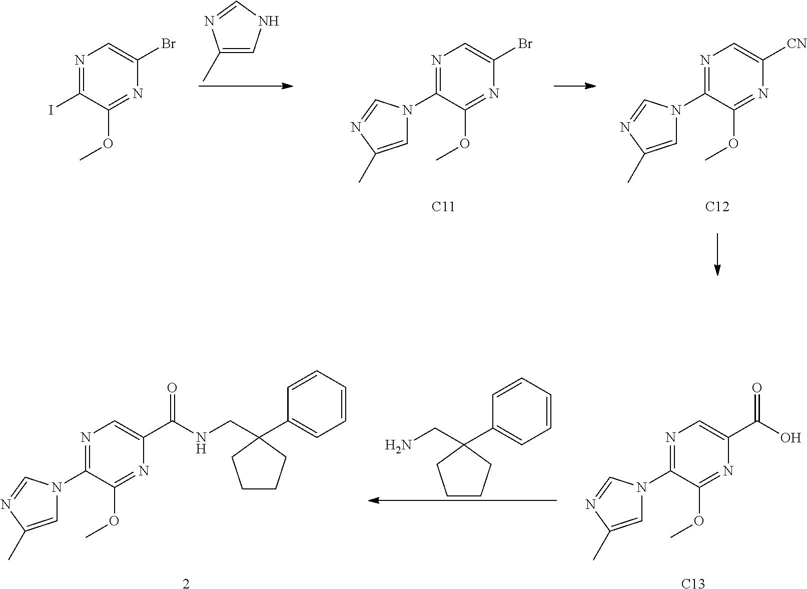

Step 1. Synthesis of 5-bromo-3-methoxy-2-(4-methyl-1H-imidazol-1-yl)pyrazine (C11). A solution of 5-bromo-2-iodo-3-methoxypyrazine (which may be prepared according to Garg, N. K. et al., J. Am. Chem. Soc. 2002, 124, 13179-13184) (92 g, 0.29 mol), 4-methyl-1H-imidazole (38.5 g, 0.47 mol), K₃PO₄ (157 g, 0.74 mol) and trans-1,2-diaminocyclohexane (15 mL, 0.12 mol) in dioxane (300 mL) was heated at reflux under a stream of argon for 15 minutes. Copper(I) iodide (5.5 g, 29 mmol) was added, and the reaction mixture was heated at reflux for an additional 30 minutes. After cooling to room temperature, the reaction mixture was diluted with ethyl acetate (1.0 L) and chromatographed on silica (Gradient: 0% to 16% methanol in ethyl acetate) to provide the title compound. Yield: 21.7 g, 0.081 mol, 28%. ¹H NMR (400 MHz, CDCl₃) δ 2.28 (s, 3H), 4.15 (s, 3H), 7.53 (s, 1H), 8.08 (s, 1H), 8.38 (s, 1H).

Step 2. Synthesis of 6-methoxy-5-(4-methyl-1H-imidazol-1-yl)pyrazine-2-carbonitrile (C12). 5-Bromo-3-methoxy-2-(4-methyl-1H-imidazol-1-yl)pyrazine (C11) (16.5 g, 61.3 mmol) and zinc cyanide (17.5 g, 149 mmol) were dissolved in N,N-dimethylformamide (80 mL). The reaction flask was evacuated and filled with argon; this procedure was repeated four times more. Then tetrakis(triphenylphosphine)palladium(0) (20 g, 17 mmol) was added, and the reaction mixture was stirred at 65° C. overnight. After cooling to room temperature, the reaction mixture was poured into a mixture of water (200 mL) and concentrated aqueous ammonia (400 mL) and extracted with dichloromethane (4×200 mL). The organic layers were combined, washed with water (200 mL) and concentrated in vacuo; the residue was dissolved in toluene (200 mL) and shaken with dilute aqueous hydrochloric acid (1:4, 300 mL). The aqueous layer was washed with toluene (300 mL), neutralized with saturated aqueous sodium bicarbonate solution (500 mL), and extracted with dichloromethane (2×500 mL). The combined extracts were evaporated, and the residue was chromatographed on silica (Eluant: toluene/ethyl acetate mixture) to provide the title compound. Yield: 5.7 g, 26 mmol, 42%. ¹H NMR (400 MHz, DMSO-d₆) δ 2.18 (s, 3H), 4.10 (s, 3H), 7.69 (s, 1H), 8.50 (s, 1H), 8.70 (s, 1 H).

Step 3. Synthesis of 6-methoxy-5-(4-methyl-1H-imidazol-1-yl)pyrazine-2-carboxylic acid, trifluoroacetate salt (C13). 6-Methoxy-5-(4-methyl-1H-imidazol-1-yl)pyrazine-2-carbonitrile (C12) was dissolved in concentrated hydrochloric acid (50 mL). The resulting solution was kept at 60° C. for 30 minutes, cooled to room temperature, filtered and concentrated in vacuo. The residue was dissolved in a mixture of acetic acid (100 mL) and acetic anhydride (200 mL), cooled to 0° C., and sodium nitrite (16.0 g, 0.23 mol) was added portion-wise. The reaction mixture was stirred at room temperature overnight and evaporated. The residue was treated with acetic anhydride (20 mL) and dichloromethane (100 mL) and filtered; the filtrate was concentrated in vacuo. The residue was dissolved in a mixture of acetic acid (50 mL) and water (200 mL), and the mixture evaporated. The residue was subjected to preparative HPLC to afford the title product (1.5 g, 6.4 mmol). LCMS m/z 235.1 (M+1). ¹H NMR (400 MHz, DMSO-d₆) δ 2.33 (s, 3H), 4.14 (s, 3H), 8.04 (s, 1H), 8.73 (s, 1H), 9.48 (s, 1H), 10.8 (br s, 2H).

Step 4. Synthesis of 6-methoxy-5-(4-methyl-1H-imidazol-1-yl)-N-[(1-phenylcyclopentyl)methyl]pyrazine-2-carboxamide (2). 6-Methoxy-5-(4-methyl-1H-imidazol-1-yl)pyrazine-2-carboxylic acid, trifluoroacetate salt (C13) (50 mg, 0.14 mmol), 1-(1-phenylcyclopentyl)methanamine (this compound may be prepared according to the method of R. Hadida et al., PCT Int. Appl., WO 2005035514 A2 Apr. 21, 2005) (25.2 mg, 0.144 mmol), 1H-benzotriazol-1-ol (29.2 mg, 0.216 mmol) and N-[3-(dimethylamino)propyl]-N′-ethylcarbodiimide hydrochloride (41.4 mg, 0.216 mmol) were combined in N,N-dimethylformamide. Diisopropylethylamine (0.10 mL, 0.57 mmol) was added and the reaction mixture was stirred at room temperature for 18 hours. The reaction mixture was diluted with ethyl acetate (40 mL) and washed with saturated aqueous sodium carbonate solution (20 mL), water (20 mL) and brine (20 mL), then dried over magnesium sulfate, filtered and concentrated under reduced pressure. The residue was triturated with dimethyl sulfoxide to provide the title product as a white solid. Yield: 10 mg, 0.026 mmol, 19%. LCMS m/z 392.6 (M+1). ¹H NMR (400 MHz, CDCl₃) δ 1.74-1.83 (m, 2H), 1.89-1.98 (m, 4H), 2.00-2.08 (m, 2H), 2.29 (s, 3H), 3.61 (d, J=6.2 Hz, 2H), 3.91 (s, 3H), 7.16 (br t, J=6 Hz, 1H), 7.24-7.30 (m, 1H), 7.35-7.38 (m, 4H), 7.63-7.65 (m, 1H), 8.49 (br s, 1H), 8.76 (br s, 1H).

### Example 3

### N-{[1-(4-Chlorophenyl)cyclopropyl]methyl}-6-methoxy-5-(4-methyl-1H-imidazol-1-yl)pyridine-2-carboxamide (3)

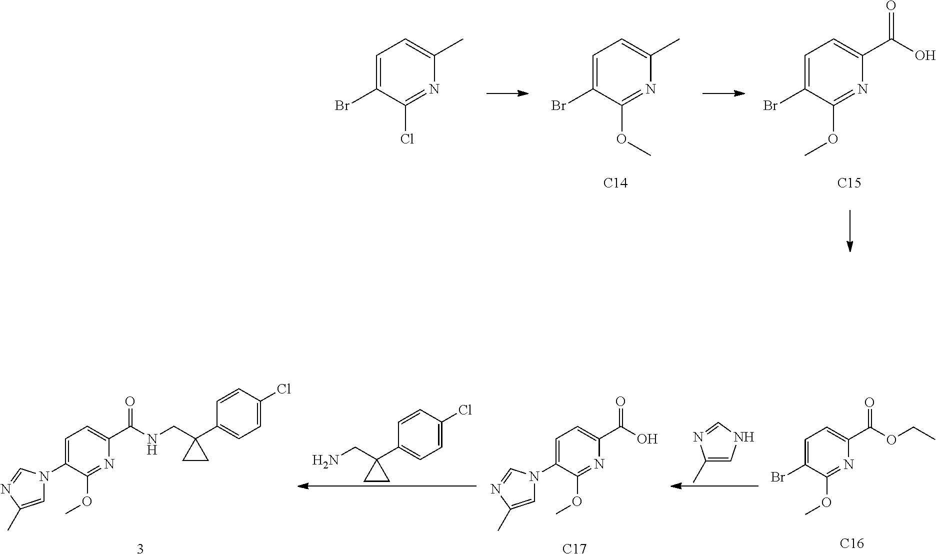

Step 1. Synthesis of 3-bromo-2-methoxy-6-methylpyridine (C14). A mixture of 3-bromo-2-chloro-6-methylpyridine (75.4 g, 0.365 mol) and sodium methoxide (59.1 g, 1.1 mol) in absolute methanol (700 mL) was heated at reflux for 5 days. Additional sodium methoxide (1 equivalent) was added, and the mixture was heated at reflux for 2 days. The solvent was removed under reduced pressure, and the residue was partitioned between water and dichloromethane. The organic layer was washed with water, dried over sodium sulfate, filtered and concentrated to provide the title product. Yield: 70.3 g, 0.348 mol, 95%.

Step 2. Synthesis of 5-bromo-6-methoxypyridine-2-carboxylic acid (C15). Selenium dioxide (72.3 g, 0.696 mol) was added to a solution of 3-bromo-2-methoxy-6-methylpyridine (C14) (70.3 g, 0.348 mol) in Dowtherm (300 mL). The reaction mixture was heated at 200° C. for 3 hours; after cooling to room temperature, the mixture was diluted with ethyl acetate and filtered through Celite. The filtrate was extracted twice with a cold solution of aqueous potassium carbonate. The combined aqueous layers were acidified to pH 5, and the resulting precipitate was isolated by filtration to provide the title product. Yield: 12.2 g, 0.0526 mol, 15%.

Step 3. Synthesis of ethyl 5-bromo-6-methoxypyridine-2-carboxylate (C16). para-Toluenesulfonic acid hydrate (roughly 0.3 g) was added to a solution of 5-bromo-6-methoxypyridine-2-carboxylic acid (C15) (12.2 g, 52.6 mmol) in ethanol (300 mL). The reaction mixture was heated at reflux for 48 hours, then concentrated in vacuo to provide the title product. Yield: 13.5 g, 51.9 mmol, 99%.

Step 4. Synthesis of 6-methoxy-5-(4-methyl-1H-imidazol-1-yl)pyridine-2-carboxylic acid (C17). A mixture of ethyl 5-bromo-6-methoxypyridine-2-carboxylate (C16) (13.5 g, 51.9 mmol), 4-methyl-1H-imidazole (5.96 g, 72.6 mmol), copper(I) iodide (1.97 g, 10.3 mmol) and cesium carbonate (33.83 g, 103.8 mmol) in N,N-dimethylformamide (100 mL) was heated at 120° C. for 48 hours under a flow of argon, then concentrated in vacuo. The residue was diluted with water and filtered through Celite. After concentration of the filtrate, the residue was purified by reverse-phase chromatography (Eluants: water, then 30% aqueous acetonitrile), then dissolved in water (10 mL) and precipitated by addition of methanol (10 mL); filtration provided the title product. Yield: 1.52 g, 6.52 mmol, 13%. LCMS m/z 234.1 (M+1). ¹H NMR (400 MHz, D₂O) δ 2.41 (s, 3H), 4.09 (s, 3H), 7.59 (br s, 1H), 7.71 (d, J=7.8 Hz, 1H), 7.99 (d, J=7.8 Hz, 1H), 9.05 (br s, 1H).

Step 5. Synthesis of N-{[1-(4-chlorophenyl)cyclopropyl]methyl}-6-methoxy-5-(4-methyl-1H-imidazol-1-yl)pyridine-2-carboxamide (3). The title compound was prepared from 6-methoxy-5-(4-methyl-1H-imidazol-1-yl)pyridine-2-carboxylic acid (C17) and 1-[1-(4-chlorophenyl)cyclopropyl]methanamine (which can be prepared by the general method of L. M. Salter-Cid et at., PCT Int. Appl. WO 2006094201 A2, Sep. 8, 2006) according to the general procedure for the synthesis of 6-methoxy-5-(4-methyl-1H-imidazol-1-yl)-N-[(1-phenylcyclopentyl)methyl]pyrazine-2-carboxamide (2) in Example 2, except that the crude product in this case was purified by chromatography on silica (Gradient: 0% to 70% [10% (2 N ammonia in methanol): 90% ethyl acetate] in ethyl acetate) to provide the title product as a foamy solid. Yield: 183 mg, 0.461 mmol, 93%. LCMS m/z 397.5, 399.5 (M+1). ¹H NMR (400 MHz, CDCl₃) δ 0.85 (br s, 2H), 0.95 (br s, 2H), 2.24 (s, 3H), 3.58 (d, J=5.7 Hz, 2H), 3.89 (s, 3H), 6.96 (s, 1H), 7.25 (AB quartet, J_{AB}=8.2 Hz, Δν_{AB}=20.9 Hz, 4H), 7.63 (d, J=7.8 Hz, 1 H), 7.71 (br s, 1 H), 7.79-7.82 (m, 2H).

### Example 4

### 6-Methoxy-5-(4-methyl-1H-imidazol-1-yl)-N-[3-(trifluoromethyl)benzyl]pyrazine-2-carboxamide (4)

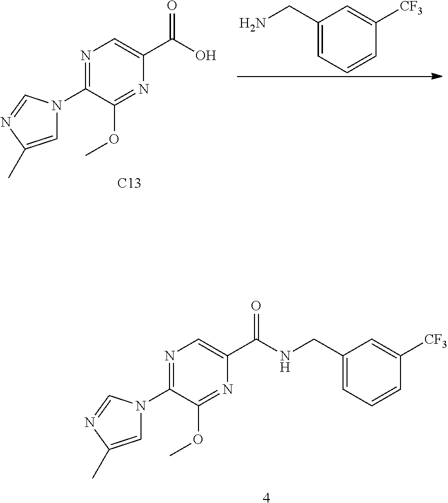

The title product was prepared according to the general procedure for the synthesis of 6-methoxy-5-(4-methyl-1H-imidazol-1-yl)-N-[(1-phenylcyclopentyl)methyl]pyrazine-2-carboxamide (2) in Example 2, except that 1-[3-(trifluoromethyl)phenyl]methanamine was used in place of 1-(1-phenylcyclopentyl)methanamine. The product was obtained as a white solid. Yield: 10 mg, 0.026 mmol, 19%. LCMS m/z 392.5 (M+1). ¹H NMR (400 MHz, CDCl₃) δ 2.31 (s, 3H), 4.18 (s, 3H), 4.77 (d, J=6.2 Hz, 2H), 7.48-7.52 (m, 1H), 7.57-7.63 (m, 3H), 7.68-7.69 (m, 1H), 7.80 (br t, J=6 Hz, 1H), 8.54 (s, 1H), 8.87 (s, 1H).

### Example 5

### N-{[1-(4-Fluorophenyl)cyclopropyl]methyl}-6-methoxy-5-(4-methyl-1H-imidazol-1-yl)pyridine-2-carboxamide (5)

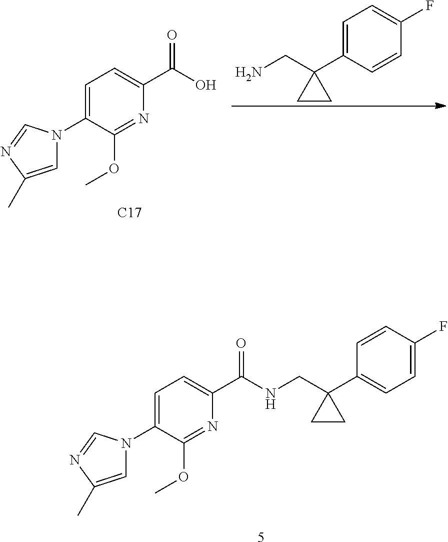

The title compound was prepared according to the general procedure for the synthesis of N-{[1-(4-chlorophenyl)cyclopropyl]methyl)-6-methoxy-5-(4-methyl-1H-imidazol-1-yl)pyridine-2-carboxamide (3) in Example 3, except that 1-[1-(4-fluorophenyl)cyclopropyl]methanamine was used in place of 1-[1-(4-chlorophenyl)cyclopropyl]methanamine. The product was obtained as a white solid. Yield: 176 mg, 0.463 mmol, 54%. LCMS m/z 381.6 (M+1). ¹H NMR (400 MHz, CDCl₃) δ 0.80-0.83 (m, 2H), 0.90-0.93 (m, 2H), 2.22 (d, J=0.9 Hz, 3H), 3.54 (d, J=6.0 Hz, 2H), 3.88 (s, 3H), 6.90-6.95 (m, 3H), 7.30 (dd, J=8.8, 5.4 Hz, 2H), 7.61 (d, J=7.8 Hz, 1H), 7.72 (br t, J=6 Hz, 1H), 7.78 (s, 1H), 7.79 (d, J=7.8 Hz, 1H).

### Example 6

### N-{[1-(4-Fluorophenyl)cyclopropyl]methyl}-6-methoxy-5-(4-methyl-1H-imidazol-1-yl)pyrazine-2-carboxamide (6)

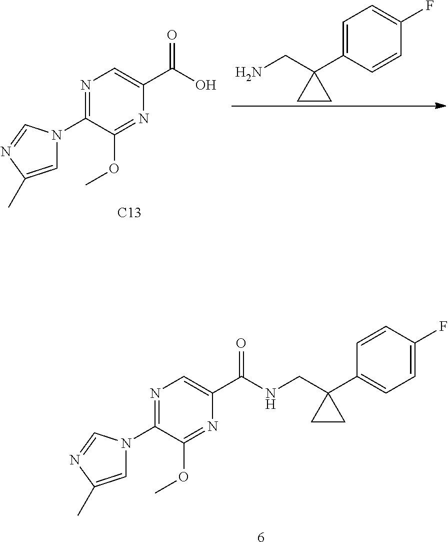

The title compound was prepared according to the general procedure for the synthesis of N-{[1-(4-fluorophenyl)cyclopropyl]methyl}-6-methoxy-5-(4-methyl-1H-imidazol-1-yl)pyridine-2-carboxamide (5) in Example 5, except that 6-methoxy-5-(4-methyl-1H-imidazol-1-yl)pyrazine-2-carboxylic acid, trifluoroacetate salt (C13) was used in place of 6-methoxy-5-(4-methyl-1H-imidazol-1-yl)pyridine-2-carboxylic acid (C17). The product was obtained as a white foam. Yield: 157 mg, 0.412 mmol, 96%. LCMS m/z 382.5 (M+1). NMR (400 MHz, CDCl₃) δ 0.89-0.92 (m, 2H), 0.98-1.01 (m, 2H), 2.30 (d, J=1.0 Hz, 3H), 3.62 (d, J=6.0 Hz, 2H), 4.06 (s, 3H), 7.01 (dd, J=8.7, 8.7 Hz, 2H), 7.36 (dd, J=8.8, 5.3 Hz, 2H), 7.44 (br t, J=5.5 Hz, 1 H), 7.66 (m, 1H), 8.51 (d, J=1.0 Hz, 1H), 8.77 (s, 1H).

### Example 7

### N-{[1-(4-Chlorophenyl)cyclopropyl]methyl}-6-methoxy-5-(4-methyl-1H-imidazol-1-yl)pyrazine-2-carboxamide (7)

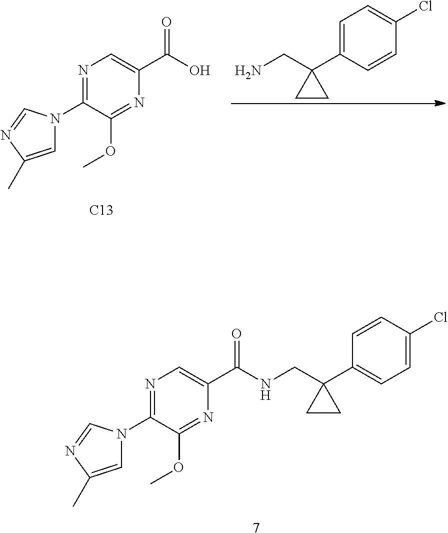

The title compound was prepared according to the general procedure for the synthesis of N-{[1-(4-chlorophenyl)cyclopropyl]methyl}-6-methoxy-5-(4-methyl-1H-imidazol-1-yl)pyridine-2-carboxamide (3) in Example 3, except that 6-methoxy-5-(4-methyl-1H-imidazol-1-yl)pyrazine-2-carboxylic acid, trifluoroacetate salt (C13) was used in place of 6-methoxy-5-(4-methyl-1H-imidazol-1-yl)pyridine-2-carboxylic acid (C17). The product was obtained as a foamy white solid. Yield: 152 mg, 0.382 mmol, 89%. LCMS m/z 398.5, 400.5 (M+1). ¹H NMR (400 MHz, CDCl₃) δ 0.90-0.93 (m, 2H), 1.00-1.02 (m, 2H), 2.30 (d, J=1.0 Hz, 3H), 3.63 (d, J=6.0 Hz, 2H), 4.07 (s, 3H), 7.28-7.34 (m, 4H), 7.43 (br t, J=5.5 Hz, 1H), 7.66 (m, 1H), 8.51 (d, J=1.2 Hz, 1H), 8.77 (s, 1H).

Biological data for Examples 1-7 is provided in Table 2. Data was obtained either on the compound as a free base or on a pharmaceutically acceptable salt of the compound.

### Examples 8-47

### Methods

Method A: Amide coupling with N-[3-(dimethylamino)propyl]-N′-ethylcarbodiimide hydrochloride

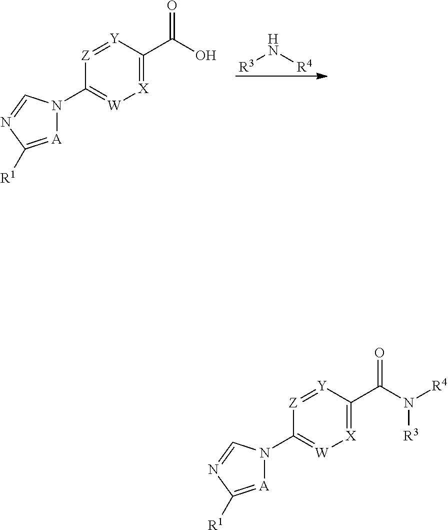

The appropriate heteroaryl carboxylic acid from one of the foregoing Preparations or Examples (1 equivalent), an amine HNR³R⁴ (1.03 equivalents), 1H-benzotriazol-1-ol (1.5 equivalents) and N-[3-(dimethylamino)propyl]-N′-ethylcarbodiimide hydrochloride (1.5 equivalents) were combined in N,N-dimethylformamide (0.1 M in substrate). Diisopropylethylamine (4 equivalents) was added and the reaction mixture was stirred at room temperature for 18 hours. The reaction mixture was diluted with ethyl acetate and washed with saturated aqueous sodium bicarbonate solution, water and brine, then dried over magnesium sulfate, filtered and concentrated under reduced pressure. Purification was carried out by preparative HPLC (Column: Waters Sunfire C₁₈, 5 μm) using one of the following gradient systems: 1) Mobile phase A: 0.05% TFA in water (v/v); Mobile phase B: 0.05% TFA in acetonitrile (v/v); 15% to 100% B; or 2) Mobile phase A: 0.05% formic acid in water (v/v); Mobile phase B: 0.05% formic acid in acetonitrile (v/v); 10% to 100% B. See Table 1 for characterization data; biological activity is provided in Table 2.

Method B: Amide coupling with O-(7-azabenzotriazol-1-yl)-N,N,N′N′-tetramethyluronium hexafluorophosphate

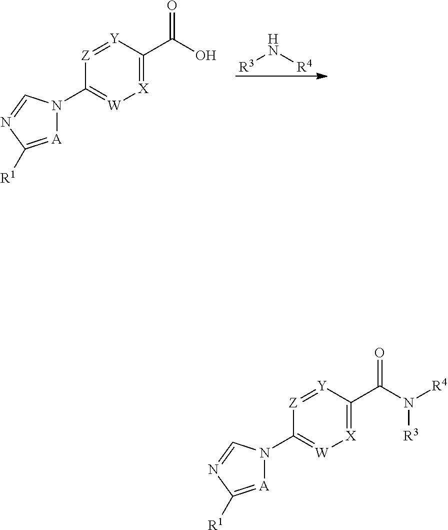

The appropriate heteroaryl carboxylic acid from one of the foregoing Preparations or Examples (1 equivalent), an amine HNR³R⁴ (1 equivalent), and O-(7-azabenzotriazol-1-yl)-N,N,N′,N′-tetramethyluronium hexafluoro-phosphate (1.1 equivalents) were combined in N,N-dimethylformamide (0.1 M in substrate). Diisopropylethylamine (2-4 equivalents) was added and the reaction mixture was stirred at room temperature for 18 hours. The reaction mixture was filtered and purified by preparative HPLC using one of the following systems: 1) Column: Waters Sunfire C₁₈, 5 μm; Mobile phase A: 0.05% TFA in water (v/v); Mobile phase B: 0.05% TFA in acetonitrile (v/v); 10% to 100% B; or 2) Column: Waters XBridge C₁₈, 5 μm; Mobile phase A: 0.03% NH₄OH in water (v/v); Mobile phase B: 0.03% NH₄OH in acetonitrile (v/v); 20% to 100% B. See Table 1 for characterization data; biological activity is provided in Table 2.

TABLE 1
Characterization data for examples 8-47.

| | | | | Retention | |
|---|---|---|---|---|---|
| | | | | Time | |
| | | | | (min) | Mass |
| | | | | [HPLC | spectrum: |
| | | | | methods | Observed |
| Ex | | | | in | ion m/z |
| # | Method | Structure | IUPAC Name | footnotes] | (M + 1) |
| 8 | A⁶ | 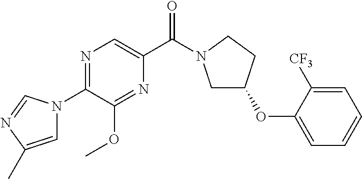 | 3-methoxy-2-(4-methyl- 1H-imidazol-1-yl)-5- ({(3S)-3-[2- (trifluoromethyl)- phenoxy]pyrrolidin-1- yl}carbonyl)pyrazine | 1.67¹ | 448.0 |
| 9 | A⁶ | 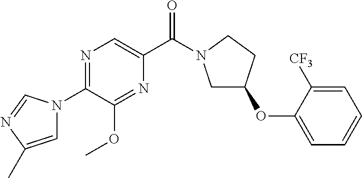 | 3-methoxy-2-(4-methyl- 1H-imidazol-1-yl)-5- ({(3R)-3-[2- (trifluoromethyl)- phenoxy]pyrrolidin-1- yl}carbonyl)pyrazine | 1.69¹ | 448.1 |
| 10 | A⁷ | 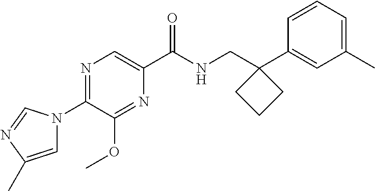 | 6-methoxy-5-(4-methyl- 1H-imidazol-1-yl)-N- {[1-(3- methylphenyl) cyclobutyl]- methyl}pyrazine-2- carboxamide | 2.33¹ | 392.2 |
| 11 | A⁷ | 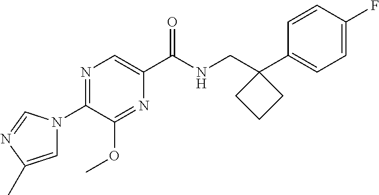 | N-{[1-(4-fluorophenyl)- cyclobutyl]methyl}-6- methoxy-5-(4-methyl- 1H-imidazol-1- yl)pyrazine-2- carboxamide | 1.84¹ | 396.1 |
| 12 | A⁸ | 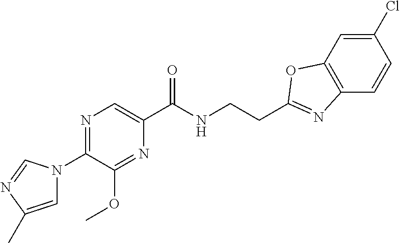 | N-[2-(6-chloro-1,3- benzoxazol-2- yl)ethyl]-6-methoxy-5- (4-methyl-1H- imidazol-1- yl)pyrazine-2- carboxamide | 1.82¹ | 413.0, 415.0 |
| 13 | A | 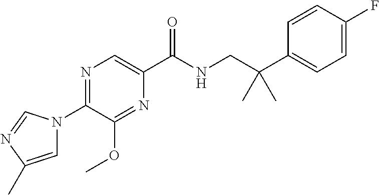 | N-[2-(4-fluorophenyl)-2- methylpropyl]-6- methoxy-5-(4-methyl- 1H-imidazol-1- yl)pyrazine-2- carboxamide | 2.07¹ | 384.2 |
| 14 | A⁹ | 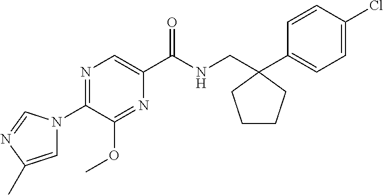 | N-{[1-(4- chlorophenyl) cyclopentyl] methyl}-6-methoxy- 5-(4-methyl-1H- imidazol-1- yl)pyrazine-2- carboxamide | 2.12¹ | 426.1, 428.0 |
| 15 | A¹⁰ | 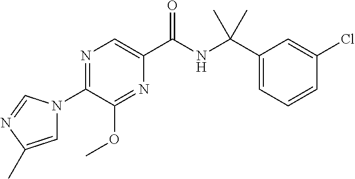 | N-[1-(3-chlorophenyl)- 1-methylethyl]-6- methoxy-5-(4-methyl- 1H-imidazol-1- yl)pyrazine-2- carboxamide | 2.11¹ | 386.1, 388.1 |
| 16 | B¹¹ | 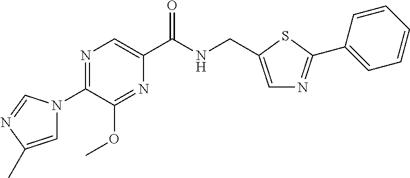 | 6-methoxy-5-(4-methyl- 1H-imidazol-1-yl)-N- [(2-phenyl-1,3-thiazol- 5-yl)methyl]pyrazine- 2-carboxamide | 1.89² | 407.0 |
| 17 | A¹⁰ | 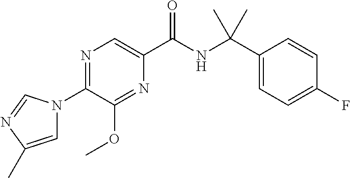 | N-[1-(4-fluorophenyl)-1- methylethyl]-6- methoxy-5-(4-methyl- 1H-imidazol-1- yl)pyrazine-2- carboxamide | 1.99² | 370.1 |
| 18 | A | 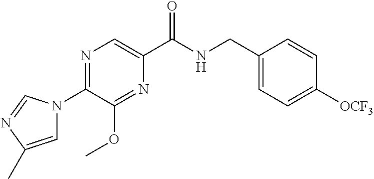 | 6-methoxy-5-(4-methyl- 1H-imidazol-1-yl)-N- [4- (trifluoromethoxy) benzyl]- pyrazine-2- carboxamide | 1.79¹ | 408.0 |
| 19 | A | 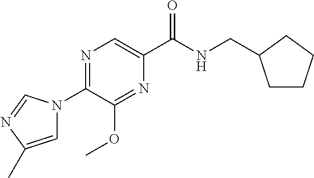 | N-(cyclopentylmethyl)- 6-methoxy-5-(4- methyl-1H-imidazol-1- yl)pyrazine-2- carboxamide | 1.70¹ | 316.2 |
| 20 | A⁶ | 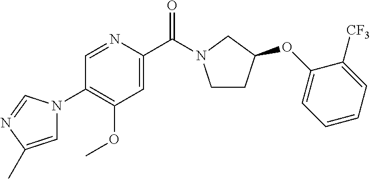 | 4-methoxy-5-(4-methyl- 1H-imidazol-1-yl)-2- ({(3S)-3-[2- (trifluoromethyl)- phenoxy]pyrrolidin-1- yl}carbonyl)pyridine | 1.14³ | 447.65 |
| 21 | A | 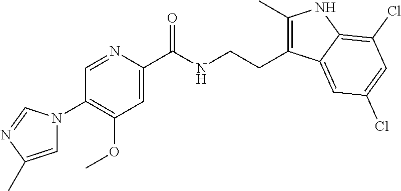 | N-[2-(5,7-dichloro-2- methyl-1H-indol-3- yl)ethyl]-4-methoxy-5- (4-methyl-1H- imidazol-1-yl)pyridine- 2-carboxamide | 1.31³ | 458.15 |
| 22 | A¹² | 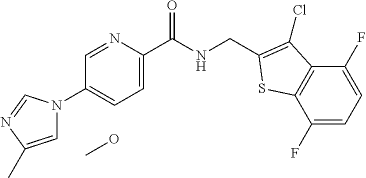 | N-[(3-chloro-4,7- difluoro-1-benzothien- 2-yl)methyl]-4- methoxy-5-(4-methyl- 1H-imidazol-1- yl)pyridine-2- carboxamide | 1.32³ | 449.35 |
| 23 | A¹³ | 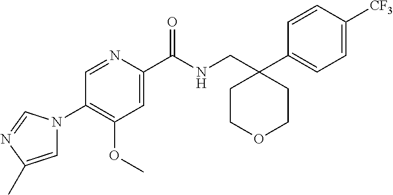 | 4-methoxy-5-(4-methyl- 1H-imidazol-1-yl)-N- ({4-[4- (trifluoromethyl)phenyl]- tetrahydro-2H-pyran- 4-yl}methyl)pyridine-2- carboxamide | 1.16³ | 475.55 |
| 24 | A | 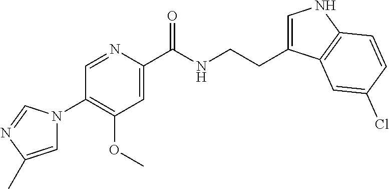 | N-[2-(5-chloro-1H- indol-3-yl)ethyl]-4- methoxy-5-(4-methyl- 1H-imidazol-1- yl)pyridine-2- carboxamide | 1.10³ | 410.15 |
| 25 | B⁹ | 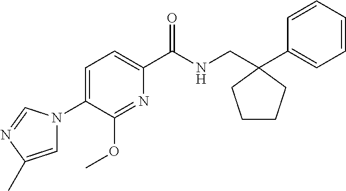 | 6-methoxy-5-(4-methyl- 1H-imidazol-1-yl)-N- [(1- phenylcycloentyl) methyl] pyridine-2- carboxamide | 2.13² | 391.1 |
| 26 | B⁸ | 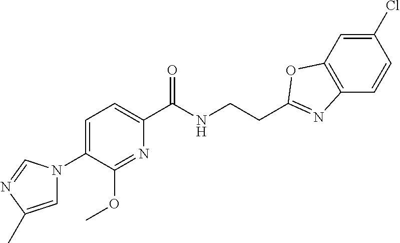 | N-[2-(6-chloro-1,3- benzoxazol-2- yl)ethyl]-6-methoxy-5- (4-methyl-1H- imidazol-1-yl)pyridine- 2-carboxamide | 1.85² | 412.0, 414.0 |
| 27 | B⁷ | 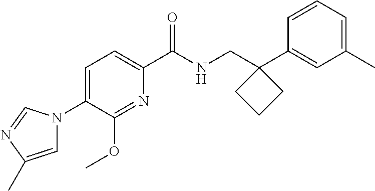 | 6-methoxy-5-(4-methyl- 1H-imidazol-1-yl)-N- {[1-(3- methylphenyl) cyclobutyl]- methyl}pyridine-2- carboxamide | 2.15² | 391.1 |
| 28 | B⁷ | 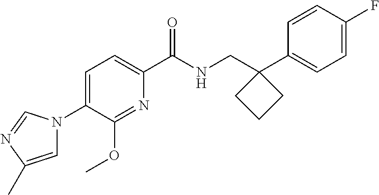 | N-{[1-(4-fluorophenyl)- cyclobutyl]methyl}-6- methoxy-5-(4-methyl- 1H-imidazol-1- yl)pyridine-2- carboxamide | 2.06² | 395.1 |
| 29 | B | 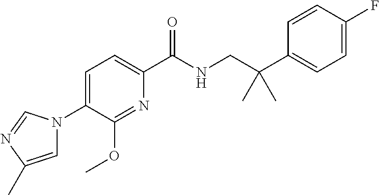 | N-[2-(4-fluorophenyl)-2- methylpropyl]-6- methoxy-5-(4-methyl- 1H-imidazol-1- yl)pyridine-2- carboxamide | 1.99² | 383.1 |
| 30 | B | 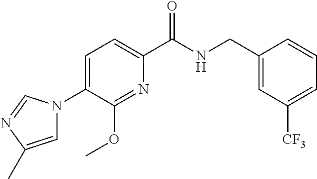 | 6-methoxy-5-(4-methyl- 1H-imidazol-1-yl)-N- [3- (trifluoromethyl) benzyl]-pyridine-2- carboxamide | 1.96² | 391.0 |
| 31 | B | 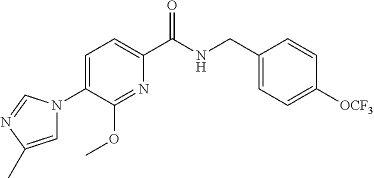 | 6-methoxy-5-(4-methyl- 1H-imidazol-1-yl)-N- [4- (trifluoromethoxy) benzyl]-pyridine-2- carboxamide | 2.05² | 407.0 |
| 32 | B¹⁰ | 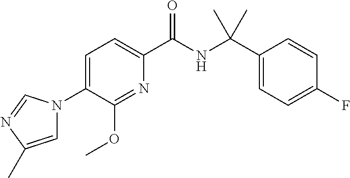 | N-[1-(4-fluorophenyl)-1- methylethyl]-6- methoxy-5-(4-methyl- 1H-imidazol-1- yl)pyridine-2- carboxamide | 1.95² | 369.1 |
| 33 | B | 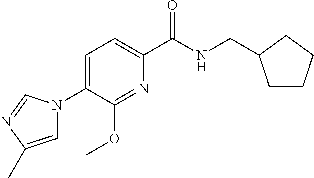 | N-(cyclopentylmethyl)- 6-methoxy-5-(4- methyl-1H-imidazol-1- yl)pyridine-2- carboxamide | 1.74² | 315.1 |
| 34 | B¹⁴ | 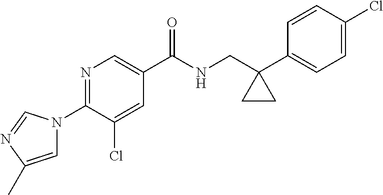 | 5-chloro-N-{[1-(4- chlorophenyl) cyclopropyl] methyl}-6-(4- methyl-1H-imidazol-1- yl)nicotinamide | 2.08¹ | 401.1 403.1 405.1 |
| 35 | B¹⁴ | 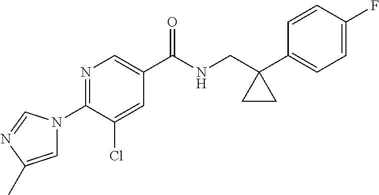 | 5-chloro-N-{[1-(4- fluorophenyl) cyclopropyl]- methyl}-6-(4- methyl-1H-imidazol-1- yl)nicotinamide | 1.93¹ | 385.1 387.1 |
| 36 | B⁶ | 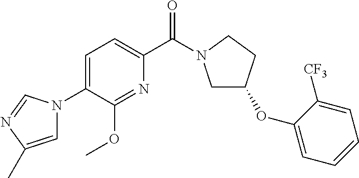 | 2-methoxy-3-(4-methyl- 1H-imidazol-1-yl)-6- ({(3S)-3-[2- (trifluoromethyl)- phenoxy]pyrrolidin-1- yl}carbonyl)pyridine | 2.49¹ | 447.1 |
| 37 | B⁶ | 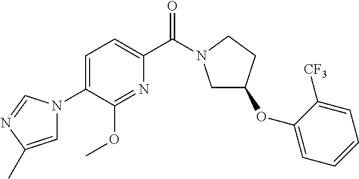 | 2-methoxy-3-(4-methyl- 1H-imidazol-1-yl)-6- ({(3R)-3-[2- (trifluoromethyl)- phenoxy]pyrrolidin-1- yl}carbonyl)pyridine | 2.48¹ | 447.1 |
| 38 | B | 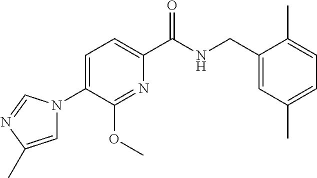 | N-(2,5-dimethylbenzyl)- 6-methoxy-5-(4- methyl-1H-imidazol-1- yl)pyridine-2- carboxamide | 1.96¹ | 351.2 |
| 39 | B¹⁵ | 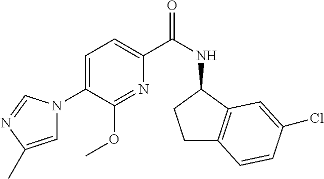 | N-[(1R)-6-chloro-2,3- dihydro-1H-inden-1- yl]-6-methoxy-5-(4- methyl-1H-imidazol-1- yl)pyridine-2- carboxamide | 2.06¹ | 383.1 385.1 |
| 40 | B¹⁵ | 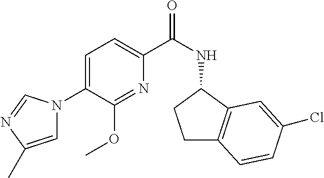 | N-[(1S)-6-chloro-2,3- dihydro-1H-inden-1- yl]-6-methoxy-5-(4- methyl-1H-imidazol-1- yl)pyridine-2- carboxamide | 2.05¹ | 383.1 385.1 |
| 41 | B | 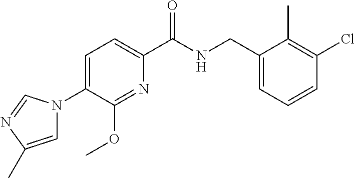 | N-(3-chloro-2- methylbenzyl)-6- methoxy-5-(4-methyl- 1H-imidazol-1- yl)pyridine-2- carboxamide | 2.62⁴ | 371.1 373.1 |
| 42 | B¹⁶ | 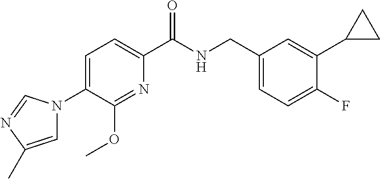 | N-(3-cyclopropyl-4- fluorobenzyl)-6- methoxy-5-(4-methyl- 1H-imidazol-1- yl)pyridine-2- carboxamide | 2.63⁴ | 381.2 |
| 43 | B | 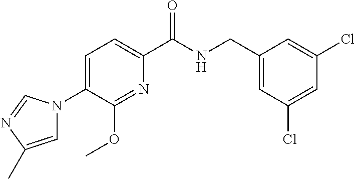 | N-(3,5-dichlorobenzyl)- 6-methoxy-5-(4- methyl-1H-imidazol-1- yl)pyridine-2- carboxamide | 2.75⁴ | 391.1 393.1 395.1 |
| 44 | B | 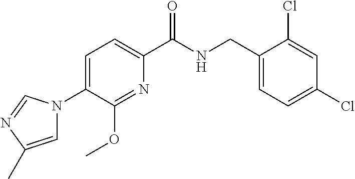 | N-(2,4-dichlorobenzyl)- 6-methoxy-5-(4- methyl-1H-imidazol-1- yl)pyridine-2- carboxamide | 2.72⁴ | 391.1 393.1 395.1 |
| 45 | B¹⁴ | 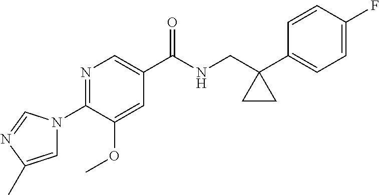 | N-{[1-(4-fluorophenyl)- cyclopropyl]methyl}-5- methoxy-6-(4-methyl- 1H-imidazol-1- yl)nicotinamide | 2.39⁴ | 381.2 |
| 46 | B⁶ | 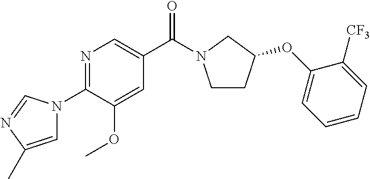 | 3-methoxy-2-(4-methyl- 1H-imidazol-1-yl)-5- ({(3R)-3-[2- (trifluoromethyl)- phenoxy]pyrrolidin-1- yl}carbonyl)pyridine | 2.56⁵ | 447.1 |
| 47 | B⁶ | 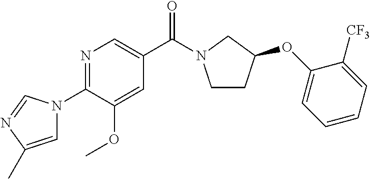 | 3-methoxy-2-(4-methyl- 1H-imidazol-1-yl)-5- ({(3S)-3-[2- (trifluoromethyl)- phenoxy]pyrrolidin-1- yl}carbonyl)pyridine | 1.95² | 447.1 |

¹Column: Waters Atlantis dC₁₈, 4.6 × 50 mm, 5 μm; Mobile phase A: 0.05% TFA in water (v/v); Mobile phase B: 0.05% TFA in acetonitrile (v/v); Flow rate 2.0 mL/min.
Gradient:
0 minutes 5% B
4 minutes 95% B
5 minutes 95% B
²Column: Waters XBridge C₁₈, 4.6 × 50 mm, 5 μm; Mobile phase A: 0.03% NH₄OH in water (v/v); Mobile phase B: 0.03% NH₄OH in acetonitrile (v/v); Flow rate 2.0 mL/min.
Gradient:
0 minutes 10% B
4 minutes 95% B
5 minutes 95% B
³Phenomenex Gemini C₁₈, 4.6 × 50 mm, 5 μm; Mobile phase A: 0.04% formic acid, 0.01% TFA in water; Mobile phase B: 0.04% formic acid, 0.01% TFA in acetonitrile.
⁴Same HPLC conditions as footnote 1, except that 15% B was used at 0 minutes
⁵Same HPLC conditions as footnote 1, except that 10% B was used at 0 minutes
⁶The amine was prepared by a Mitsunobu reaction between an N-protected pyrrolidin-3-ol and the appropriate phenol. See K. C. Swamy et al., Chemical Reviews 2009, 109, 2551-2651.
⁷The amine may be prepared by the method of L. M. Blatt et al., PCT Int. Appl. WO 2005037214 A2, Apr. 28, 2005.
⁸The amine may be prepared by the method of P. L. Lopez-Tudanca et al., Bioorg. Med. Chem. 2003, 11, 2709-2714.
⁹The amine may be prepared by the method of R. Hadida et al., PCT Int. Appl., WO 2005035514 A2, Apr. 21, 2005.
¹⁰The amine may be prepared by the method of J. M. Elliott et al., Bioorg. Med. Chem. Lett. 2002, 12, 1755-1758.
¹¹The amine was prepared by conversion of the corresponding aldehyde to the oxime, followed by reduction with lithium aluminum hydride.
¹²(2E)-3-(2,5-Difluorophenyl)acrylic acid was converted to 3-chloro-4,7-difluoro-1-benzothiophene-2-carbonyl chloride according to the general method of J. A. Kaizerman et al., J. Med. Chem. 2003, 46, 3914-3929. Transformation of the acid chloride moiety into an aminomethyl group was carried out by treatment with ammonia followed by sodium borohydride/BF₃ etherate reduction.
¹³The amine can be prepared using chemistry similar to that described for 1-{4-[3-(trifluoromethyl)phenyl]tetrahydro-2H-pyran-4-yl}methanamine (C9) in Example 1.
¹⁴The amine may be prepared by the general method of L. M. Salter-Cid et al., PCT Int. Appl. WO 2006094201 A2, Sep. 8, 2006.
¹⁵The amine was prepared from the corresponding ketone according to the general method of J. T. Colyer et al., J. Org. Chem. 2006, 71, 6859-6862.
¹⁶Ethyl 3-bromo-4-fluorobenzoate was converted to the cyclopropyl derivative by the method of H. Haning et al., Bioorg. Med. Chem. Lett. 2005, 15, 1835-1840. Lithium hydroxide-mediated hydrolysis of the ester provided the corresponding carboxylic acid, which was converted to the primary amide and reduced with lithium aluminum hydride.

### Cell-Based γ-Secretase Assay with ELISA Readout

The ability of compounds to modulate production of amyloid beta protein Aβ (1-42) was determined using human WT-APP overexpressing CHO cells. Cells were plated at 22,000 cells/100 μL well in 96 well tissue culture treated, clear plates (Falcon) in DMEM/F12 based medium and incubated for 24 hours at 37° C. Compounds for testing were diluted in 100% DMSO to achieve an eleven points, half log, dose response for IC₅₀ determinations. Compounds were added in fresh medium to achieve 1% final DMSO. Appropriate vehicle and inhibitor controls were added to obtain maximum and minimum inhibition values for the assay before the plates were incubated for about 24 hours at 37° C.

Coating of ELISA assay plates was initiated by addition of 50 μL/well of an in-house Aβ (1-42) specific antibody at (4 μg/mL) in 0.1 M NaHCO₃ (pH 9.0) into black 384-well Maxisorp® plates (Nunc) and incubated overnight at 4° C. The capture antibody was then aspirated from the ELISA assay plates and 100 μL/well of Blocking Buffer (Dulbecco's PBS, 1.5% BSA (Sigma A7030)) added. Ambient temperature incubation was allowed to proceed for a minimum of two hours before washing 2×100 μL with Wash Buffer (Dulbecco's PBS, 0.05% Tween 20). Assay Buffer (Dulbecco's PBS, 1.0% BSA (Sigma A7030), 0.05% Tween 20) 20 μL/well was then added.

After incubation overnight at 37° C., 5% CO₂, 40 μL (in duplicate) of experimental conditioned media were transferred into wells of the blocked ELISA plates containing the capture antibody, followed by overnight incubation at 4° C. Cell toxicity was measured in the corresponding cells after removal of the media for the Aβ (1-42) assay by a colorimetric cell proliferation assay (CellTiter 96® AQᵤₑₒᵤₛ One Solution Cell Proliferation Assay, Promega) according to the manufacturer's instructions.

After overnight incubation of the ELISA assay plates at 4° C., unbound Aβ peptides were removed thorough (4×100 μL) washes with Wash Buffer. Europium (Eu) labeled (custom labeled, Perkin Elmer) Aβ (1-16) 6e10 Monoclonal Antibody (Covance #SIG-39320 was added, (50 μL /well Eu-6e10 @ 1:5000, 20 uM EDTA) in Assay Buffer. Incubation at ambient temperature for a minimum of 2 hours was followed by (4×100 μL) washes with Wash Buffer, before 50 μL/well of Delfia Enhancement Solution (Perkin Elmer) was added. Following a one hour ambient temperature incubation, the plates were read on an EnVision plate reader (Perkin Elmer) using standard DELFIA TRF settings. Data analysis including inhibitory IC₅₀ determination was performed using nonlinear regression fit analysis (in-house software) and the appropriate plate mean values for the maximum and minimum inhibition controls.

TABLE 2
Biological data for examples 1-47.

| | Aβ 42B IC₅₀ |
|---|---|
| | (μM) (Geometric |
| | Mean of 2-8 |
| Example | Determinations) |
| 1 | 13.6 |
| 2 | 2.29 |
| 3 | 0.186 |
| 4 | 1.75 |
| 5 | 0.34 |
| 6 | 1.30 |
| 7 | 0.591 |
| 8 | 0.535 |
| 9 | 0.643 |
| 10 | 0.827 |
| 11 | 1.38 |
| 12 | 1.43 |
| 13 | 1.36 |
| 14 | 1.45 |
| 15 | 1.82 |
| 16 | 1.94 |
| 17 | 2.41 |
| 18 | 5.43 |
| 19 | 5.77 |
| 20 | 5.33 |
| 21 | 6.25 |
| 22 | >15.8 |
| 23 | 17.2 |
| 24 | 18.0 |
| 25 | 0.316 |
| 26 | 0.524 |
| 27 | 0.438 |
| 28 | 0.314 |
| 29 | 0.502 |
| 30 | 0.502 |
| 31 | 1.01 |
| 32 | 2.01 |
| 33 | 0.726 |
| 34 | 1.37 |
| 35 | 5.77 |
| 36 | 0.207 |
| 37 | 0.285 |
| 38 | 0.667 |
| 39 | 1.34 |
| 40 | 0.259 |
| 41 | 0.332 |
| 42 | 0.319 |
| 43 | 0.216 |
| 44 | 0.394 |
| 45 | 2.23 |
| 46 | 2.30 |
| 47 | 3.44 |

The compounds in Table 3 were prepared using Method B. The amine coupling partners were prepared according to the chemistry described in the Preparations section, or are readily prepared using chemistry well known to one skilled in the art.

TABLE 3

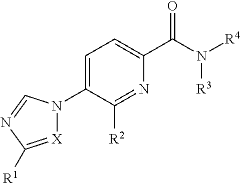

| | | | | Mass | |
|---|---|---|---|---|---|
| | | | | spec: | |
| | | | | observed | |
| | | | | ion m/z | |
| | | | | (M + 1); | |
| | | Aβ 42 | | HPLC | ¹H NMR (400 MHz, CDCl₃ |
| | | IC₅₀ | | reten- | unless otherwise specified); |
| Ex# | Structure of NR³R⁴ | (μM)* | IUPAC Name | tion time | observed peaks, δ (ppm) |
| 48 | 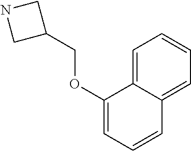 | 0.096 | 2-methoxy-3-(4- methyl-1H- imidazol-1-yl)-6- ({3-[(1- naphthyloxy) methyl]azetidin-1- yl}carbonyl) pyridine | 429.1, 2.62 min^{A} | |
| 49^{B} | 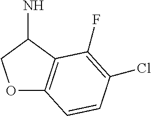 | 0.194 | N-[5-chloro-4- fluoro-2,3- dihydro-1- benzofuran-3- yl]-6-methoxy-5- (4-methyl-1H- imidazol-1- yl)pyridine-2- carboxamide | 403.2 | 2.31 (d, J = 0.9 Hz, 3H), 4.03 (s, 3H), 4.57 (dd, J = 10.2, 4.3 Hz, 1H), 4.94 (dd, J = 10.2, 8.2 Hz, 1H), 5.98 (br ddd, J = 8, 8, 4 Hz, 1H), 6.69 (br d, J = 8.7 Hz, 1H), 7.00-7.02 (m, 1H), 7.32 (br dd, J = 8, 8 Hz, 1H), 7.72 (d, J = 7.9 Hz, 1H), 7.87 (d, J = 1.3 Hz, 1H), 7.94 (d, J = 7.9 Hz, 1H), 7.95-7.98 (m, 1H) |
| 50^{B} | 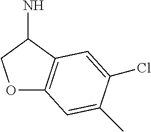 | 0.200 | N-[5-chloro-6- methyl-2,3- dihydro-1- benzofuran-3- yl]-6-methoxy-5- (4-methyl-1H- imidazol-1- yl)pyridine-2- carboxamide | 398 | ¹H NMR (400 MHz, CD₃OD), δ (ppm) 2.25 (d, J = 0.9 Hz, 3H), 2.32 (s, 3H), 4.07 (s, 3H), 4.51 (dd, J = 9.8, 5.0 Hz, 1H), 4.80 (dd, J = 9.9, 8.8 Hz, 1H), 5.81 (dd, J = 8.7, 5.0 Hz, 1H), 6.78 (s, 1H), 7.24-7.26 (m, 1H), 7.32 (s, 1H), 7.84 (d, J = 7.8 Hz, 1H), 7.95 (d, J = 7.9 Hz, 1H), 8.04 (d, J = 1.2 Hz, 1H) |
| 51^{B} | 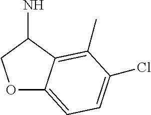 | 0.234 | N-(5-chloro-4- methyl-2,3- dihydro-1- benzofuran-3- yl)-6-methoxy-5- (4-methyl-1H- imidazol-1- yl)pyridine-2- carboxamide | 399 | 2.30 (d, J = 0.8 Hz, 3H), 2.33 (s, 3H), 3.99 (s, 3H), 4.52 (dd, J = 10.2, 3.1 Hz, 1H), 4.81 (dd, J = 10.3, 8.0 Hz, 1H), 5.88 (br ddd, J = 8, 8, 3 Hz, 1H), 6.72 (d, J = 8.6 Hz, 1H), 7.00-7.02 (m, 1H), 7.26-7.30 (m, 1H), 7.72 (d, J = 7.8 Hz, 1H), 7.81 (br d, J = 8.6 Hz, 1H), 7.85 (d, J = 1.2 Hz, 1H), 7.95 (d, J = 7.8 Hz, 1H) |
| 52^{D} | 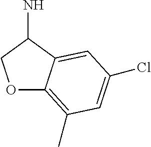 | 0.364 | N-(5-chloro-7- methyl-2,3- dihydro-1- benzofuran-3- yl)-6-methoxy-5- (4-methyl-1H- imidazol-1- yl)pyridine-2- carboxamide | 399.1 2.60 min^{A} | |
| 53^{C} | 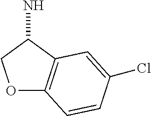 | 0.373 | N-[(3R)-5- chloro-2,3- dihydro-1- benzofuran-3- yl]-6-methoxy-5- (4-methyl-1H- imidazol-1- yl)pyridine-2- carboxamide | 385.1 | 2.29 (br s, 3H), 4.01 (s, 3H), 4.46 (dd, J = 10.2, 4.5 Hz, 1H), 4.87 (dd, J = 10.2, 8.4 Hz, 1H), 5.85 (br ddd, J = 8, 8, 4 Hz, 1H), 6.84 (d, J = 8.6 Hz, 1H), 7.01 (br s, 1H), 7.23 (br dd, J = 8.6, 2.3 Hz, 1H), 7.35 (br d, J = 2 Hz, 1H), 7.72 (d, J = 7.8 Hz, 1H), 7.86-7.91 (m, 2H), 7.94 (d, J = 7.8 Hz, 1H) |
| 54^{D} | 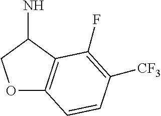 | 0.411 | N-[4-fluoro-5- (trifluoromethyl)- 2,3-dihydro-1- benzofuran-3- yl]-6-methoxy-5- (4-methyl-1H- imidazol-1- yl)pyridine-2- carboxamide | 437.1 | 2.31 (br s, 3H), 4.03 (s, 3H), 4.64 (dd, J = 10.3, 4.5 Hz, 1H), 5.00 (dd, J = 10.2, 8.4 Hz, 1H), 6.00 (br ddd, J = 8, 8, 4.5 Hz, 1H), 6.78 (d, J = 8.6 Hz, 1H), 7.01 (br s, 1H), 7.56 (br dd, J = 8, 8 Hz, 1H), 7.72 (d, J = 7.9 Hz, 1H), 7.86 (br s, 1H), 7.94 (d, J = 7.8 Hz, 1H), 8.01 (br d, J = 8 Hz, 1H) |
| 55^{B} | 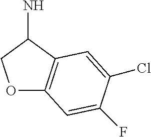 | 0.423 | N-[5-chloro-6- fluoro-2,3- dihydro-1- benzofuran-3- yl]-6-methoxy-5- (4-methyl-1H- imidazol-1- yl)pyridine-2- carboxamide | 403.2 | 2.30 (d, J = 0.8 Hz, 3H), 4.02 (s, 3H), 4.51 (dd, J = 10.2, 4.3 Hz, 1H), 4.90 (dd, J = 10.2, 8.2 Hz, 1H), 5.83 (br ddd, J = 8, 8, 4 Hz, 1H), 6.72 (d, J = 9.3 Hz, 1H), 7.00-7.02 (m, 1H), 7.40 (dd, J = 7.4, 0.6 Hz, 1H), 7.72 (d, J = 7.8 Hz, 1H), 7.85-7.89 (m, 2H), 7.94 (d, J = 7.8 Hz, 1H) |
| 56^{B} | 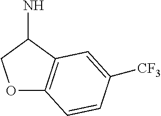 | 0.478 | 6-methoxy-5-(4- methy-1H- imidazol-1-yl)- N-[5- (trifluoromethyl)- 2,3-dihydro-1- benzofuran-3- yl]pyridine-2- carboxamide | 419.1 1.70 min^{A} | |

R¹ = methyl
R² = methoxy
X = CH
*Geometric mean of at least 2 determinations.
^{A}QC conditions: Column: Waters Atlantis dC₁₈, 4.6 × 50 mm, 5 μm; Mobile phase A: 0.05% TFA in water (v/v); Mobile phase B: 0.05% TFA in acetonitrile (v/v); Gradient: 95% H₂O/5% MeCN linear to 5% H₂O/95% MeCN over 4.0 min, Hold at 5% H₂O/95% MeCN to 5.0 min. Flow: 2.0 mL/min.
^{B}Single enantiomer, absolute stereochemistry unknown. The enantiomers of the final compound were separated using chiral prep-HPLC.
^{C}Single enantiomer, absolute stereochemistry known. This compound was prepared using 5-chloro-2,3-dihydro-1-benzofuran-3-amine enantiomer 2 (P6 enantiomer 2) from the Preparations Section. The absolute stereochemistry of this amine was determined to be (R) by single X-ray crystallography of its HCl salt.
^{D}Racemic.

We claim:

1. A compound having the structure of formula I: 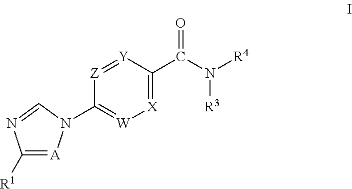
wherein A is CH;
W is CR²; X, Y, and Z are independently CH or N, and at least one of X, Y, or Z is N;
R¹ is C₁₋₆alkyl; wherein said alkyl, may be optionally substituted with one to three of fluorine, hydroxyl, or C₁₋₆alkoxy groups;
R² is hydrogen, —CF₃, cyano, halogen, C₁₋₆alkyl, or —OR⁵;
R³ and R⁴ are each independently hydrogen, C₁₋₆alkyl, C₂₋₆alkenyl, —(C(R⁶)₂)ₜ-(C₃₋₆cycloalkyl), —(C(R⁶)₂)ₜ-(4- to 10-membered heterocycloalkyl), —(C(R⁶)₂)ₜ-(C₆₋₁₀aryl), or —(C(R⁶)₂)ₜ-(5- to 10-membered heteroaryl); wherein said alkyl, alkenyl, or cycloalkyl, heterocycloalkyl, aryl, or heteroaryl moieties may be optionally independently substituted with one to three R⁶; or R³ and R⁴ together with the nitrogen to which they are bonded form a 4- to 10-membered heterocycloalkyl optionally substituted with one to three R⁶;
R⁵ is hydrogen, C₁₋₆alkyl, C₃₋₆cycloalkyl, C₃₋₆alkenyl, or C₃₋₆alkynyl; wherein said alkyl, cycloalkyl, alkenyl, or alkynyl may be optionally substituted with cyano or one to three fluorines;
each R⁶ is independently hydrogen, halogen, cyano, —CF₃, C₁₋₆alkyl, C₂₋₆alkenyl, C₂₋₆alkylidene, —(C(R⁹)₂)ₘ-(C₃₋₆cycloalkyl), —(C(R⁹)₂)ₘ-(4- to 10-membered heterocycloalkyl), —(C(R⁹)₂)ₘ-(C₆₋₁₀aryl), or —(C(R⁹)₂)ₘ-(5- to 10-membered heteroaryl), —(C(R⁹)₂)ₘ-OR⁷, —C(O)R⁷, —C(O)N(R⁷)₂, —NHC(O)R⁷, —NR⁷SO₂R⁸, or —N(R⁷)₂; wherein said C₁₋₆alkyl, C₂₋₆alkenyl, C₂₋₆alkylidene, or cycloalkyl, heterocycloalkyl, aryl, or heteroaryl moieties may be optionally independently substituted with one to three R⁹;
each R⁷ is independently hydrogen, C₁₋₆alkyl, —CF₃, —(C(R¹¹)₂)ₙ-(C₃₋₆cycloalkyl), —(C(R¹¹)₂)ₙ-(4- to 10-membered heterocycloalkyl), —(C(R¹¹)₂)ₙ-(C₆₋₁₀aryl), or —(C(R¹¹)₂)ₙ-(5- to 10-membered heteroaryl); wherein said alkyl, or cycloalkyl, heterocycloalkyl, aryl or heteroaryl moieties may be optionally independently substituted with one to three R¹¹;
each R⁸ is independently C₁₋₆alkyl, —(C(R¹²)₂)ₚ-(C₃₋₆cycloalkyl), —(C(R¹²)₂)ₚ-(4- to 10-membered heterocycloalkyl), —(C(R¹²)₂)ₚ-(C₆₋₁₀aryl), or —(C(R¹²)₂)ₚ-(5- to 10-membered heteroaryl); wherein said alkyl, or cycloalkyl, heterocycloalkyl, aryl, or heteroaryl moieties may be optionally independently substituted with one to three R¹²;
each R⁹ is independently hydrogen, C₁₋₆alkyl, C₂₋₆alkenyl, C₂₋₆alkynyl, halogen, —CF₃, —OR⁷, —(C(R¹⁰)₂)_{q}-(C₆₋₁₀aryl), or —(C(R¹⁰)₂)_{q}-(₅₋ to 10-membered heteroaryl);
each R¹⁰ is independently hydrogen, —CF₃, cyano, halogen, C₁₋₆alkyl, or —OR⁵;
each R¹¹ is independently hydrogen, C₁₋₆alkyl, C₂₋₆alkenyl, C₂₋₆alkynyl, halogen, cyano, —CF₃, or —OCF₃;
each R¹² is independently hydrogen or halogen; and
each t, m, n, p or q is an integer independently selected from 0, 1, 2, 3, and 4; and pharmaceutically acceptable salts thereof.

2-9. (canceled)

10. A compound according to claim 1 wherein R³ is hydrogen, C₁₋₆alkyl, or —(C(R⁶)₂)ₜ-(C₃₋₆cycloalkyl);
wherein said alkyl, or cycloalkyl moiety may be optionally independently substituted with one to three fluorines; and R⁴ is C₁₋₆alkyl, —(C(R⁶)₂)-(C₃₋₆cycloalkyl), —(C(R⁶)₂)-(4- to 10-membered heterocycloalkyl), —(C(R⁶)₂)ₜ-(C₆₋₁₀aryl), or —(C(R⁶)₂)ₜ-(5- to 10-membered heteroaryl); wherein said C₁-₆alkyl, or cycloalkyl, heterocycloalkyl, aryl, or heteroaryl moieties may be optionally independently substituted with one to three substituents independently selected from R⁶; or wherein R³ and R⁴ together with the nitrogen to which they are bonded form a 4- to 10-membered heterocycloalkyl, wherein said heterocycloalkyl is optionally substituted with one to three R⁶.

11. A compound according to claim 10 wherein R³ is hydrogen and R⁴ is —(C(R⁶)₂)ₜ-(C₃₋₆cycloalkyl); wherein said cycloalkyl moiety is optionally substituted with one to three R⁶.

12. A compound according to claim 10 wherein R³ is hydrogen and R⁴ is —(C(R⁶)₂)ₜ-(4- to 10-membered heterocycloalkyl); wherein said heterocycloalkyl moiety is optionally substituted with one to three R⁶.

13. A compound according to claim 10 wherein R³ is hydrogen and R⁴ is —(C(R⁶)₂)ₜ-(C₆₋₁₀aryl); wherein said aryl moiety is optionally substituted with one to three R⁶.

14. A compound according to claim 10 wherein R³ is hydrogen and R⁴ is —(C(R⁶)₂)ₜ-(5- to 10-membered heteroaryl); wherein said heteroaryl moiety is optionally substituted with one to three R⁶.

15. A compound according to claim 10 R³ and R⁴ together with the nitrogen to which they are bonded form a 4- to 10-membered heterocycloalkyl, wherein said heterocycloalkyl is optionally substituted with one to three R⁶.

16. A compound according to claim 10 wherein R³ is hydrogen, C₁₋₆alkyl, or —(C(R)₂)ₜ-(C₃₋₆cycloalkyl); wherein said alkyl or cycloalkyl moiety may be optionally independently substituted with one to three fluorines.

17. (canceled)

18. A compound according to claim 10 wherein R¹ is methyl.

19. A compound according to claim 10 wherein R² is hydrogen, halogen or —OR⁵ wherein R⁵ is hydrogen or C₁₋₆alkyl.

20. (canceled)

21. A compound according to claim 19 wherein R⁵ is methyl and a pharmaceutically acceptable salt thereof.

22. A compound selected from the group consisting of:
5-(4-methyl-1H-imidazol-1-yl)-2-({(3S)-3-[2-(trifluoromethyl)phenoxy]pyrrolidin-1-yl}carbonyl)pyridine;
2-(4-methyl-1H-imidazol-1-yl)-5-({(3S)-3-[2-(trifluoromethyl)phenoxy]pyrrolidin-1-yl}carbonyl)pyrimidine;
2-(4-methyl-1H-imidazol-1-yl)-5-({(3S)-3-[2-(trifluoromethyl)phenoxy]pyrrolidin-1-yl}carbonyl)pyrazine;
5-(4-methyl-1H-imidazol-1-yl)-2-({(3S)-3-[2-(trifluoromethyl)phenoxy]pyrrolidin-1-yl}carbonyl)pyrimidine;
3-(4-methyl-1H-imidazol-1-yl)-6-({(3S)-3-[2-(trifluoromethyl)phenoxy]pyrrolidin-1-yl}carbonyl)pyridazine;
N-(2,5-dimethylbenzyl)-5-(4-methyl-1H-imidazol-1-yl)pyridine-2- carboxamide;
N-(2,5-dimethylbenzyl)-2-(4-methyl-1H-imidazol-1-yl)pyrimidine-5-carboxamide;
N-(2,5-dimethylbenzyl)-5-(4-methyl-1H-imidazol-1-yl)pyrazine-2-carboxamide;
N-(2,5-dimethylbenzyl)-5-(4-methyl-1H-imidazol-1-yl)pyrimidine-2-carboxamide;
N-(2,5-dimethylbenzyl)-6-(4-methyl-1H-imidazol-1-yl)pyridazine-3-carboxamide;
N-{[1-(4-fluorophenyl)cyclopropyl]methyl}-5-(4-methyl-1H-imidazol-1-yl)pyridine-2-carboxamide;
N-{[1-(4-fluorophenyl)cyclopropyl]methyl}-2-(4-methyl-1H-imidazol-1-yl)pyrimidine-5-carboxamide;
N-{[1-(4-fluorophenyl)cyclopropyl]methyl}-5-(4-methyl-1H-imidazol-1-yl)pyrazine-2-carboxamide;
N-{[1-(4-fluorophenyl)cyclopropyl]methyl}-5-(4-methyl-1H-imidazol-1-yl)pyrimidine-2-carboxamide;
N-{[1-(4-fluorophenyl)cyclopropyl]methyl}-6-(4-methyl-1H-imidazol-1-yl)pyridazine-3-carboxamide;
N-[(3R)-5-chloro-2,3-dihydro-1-benzofuran-3-yl]-6-methoxy-5-(4-methyl-1H-imidazol-1-yl)pycarboxamide;
N-[(3S)-5-chloro-2,3-dihydro-1-benzofuran-3-yl]-6-methoxy-5-(4-methyl-1H-imidazol-1-yl)pycarboxamide;
N-[(3R)-7-chloro-2,3-dihydro-1-benzofuran-3-yl]-6-methoxy-5-(4-methyl-1H-imidazol-1-yl)pyridine-2-carboxamide;
N-[(3S)-7-chloro-2,3-dihydro-1-benzofuran-3-yl]-6-methoxy-5-(4-methyl-1H-imidazol-1-yl)pyridine-2-carboxamide;
N-[(3R)-6-chloro-2,3-dihydro-1-benzofuran-3-yl]-6-methoxy-5-(4-methyl-1H-imidazol-1 -yl)pycarboxamide;
N-[(3S)-6-chloro-2,3-dihydro-1-benzofuran-3-yl]-6-methoxy-5-(4-methyl-1H-imidazol-1-yl)pycridine-2-carboxamide;
N-[(3R)-4,6-dichloro-2,3-dihydro-1 -benzofuran-3-yl]-6-methoxy-5-(4-methyl-1H-imidazol-1-yl)pyridine-2-carboxamide;
N-[(3S)-4,6-dichloro-2,3-dihydro-1-benzofuran-3-yl]-6-methoxy-5-(4-methyl-1H-imidazol-1-yl)pyridine-2-carboxamide;
N-[(3R)-5,7-dichloro-2,3-dihydro-1-benzofuran-3-yl]-6-methoxy-5-(4-methyl-1H-imidazol-1-yl)pyridine-2-carboxamide;
N-[(3S)-5,7-dichloro-2,3-dihydro-1-benzofuran-3-yl]-6-methoxy-5-(4-methyl-1H-imidazol-1-yl)pyridine-2-carboxamide;
N-[(3R)-5-cyclopropyl-6-fluoro-2,3-dihydro-1-benzofuran-3-yl]-6-methoxy-5-(4-methyl-1H-imidazol-1-yl)pyridine-2-carboxamide;
N-[(3S)-5-cyclopropyl-6-fluoro-2,3-dihydro-1-benzofuran-3-yl]-6-methoxy-5-(4-methyl-1H-imidazol-1-yl)pyridine-2-carboxamide;
N-[(3R)-6-cyclopropyl-5-fluoro-2,3-dihydro-1-benzofuran-3-yl]-6-methoxy-5-(4-methyl-1H-imidazol-1-yl)pyridine-2-carboxamide;
N-[(3S)-6-cyclopropyl-5-fluoro-2,3-dihydro-1-benzofuran-3-yl]-6-methoxy-5-(4-methyl-1H-imidazol-1-yl)pyridine-2-carboxamide;
N-[(3R)-5-cyclopropyl-2,3-dihydro-1-benzofuran-3-yl]-6-methoxy-5-(4-methyl-1H-imidazol-1-yl)pyridine-2-carboxamide;
N-[(3S)-5-cyclopropyl-2,3-dihydro-1-benzofuran-3-yl]-6-methoxy-5-(4-methyl-1H-imidazol-1-yl)pyridine-2-carboxamide;
6-methoxy-5-(4-methyl-1H-imidazol-1-yl)-N-[(3R)-5-(trifluoromethyl)-2,3-dihydro-1-benzofuran-3-yl]pyridine-2-carboxamide;
6-methoxy-5-(4-methyl-1H-imidazol-1-yl)-N-[(3S)-5-(trifluoromethyl)-2,3-dihydro-1-benzofuran-3-yl]pyridine-2-carboxamide;
6-methoxy-5-(4-methyl-1H-imidazol-1-yl)-N-[(3R)-6-(trifluoromethyl)-2,3-dihydro-1-benzofuran-3-yl]pyridine-2-carboxamide;
6-methoxy-5-(4-methyl-1H-imidazol-1-yl)-N-[(3S)-6-(trifluoromethyl)-2,3-dihydro-1-benzofuran-3-yl]pyridine-2-carboxamide;
N-[(3R)-5-isopropyl-2,3-dihydro-1-benzofuran-3-yl]-6-methoxy-5-(4-methyl-1H-imidazol-1-yl)pyridine-2-carboxamide;
N-[(3S)-5-isopropyl-2,3-dihydro-1-benzofuran-3-yl]-6-methoxy-5-(4-methyl-1H-imidazol-1-yl)pyridine-2-carboxamide;
N-[(3R)-6-ethoxy-5-isopropyl-2,3-dihydro-1-benzofuran-3-yl]-6-methoxy-5-(4-methyl-1H-imidazol-1-yl)pyridine-2-carboxamide;
N-[(3S)-6-ethoxy-5-isopropyl-2,3-dihydro-1-benzofuran-3-yl]-6-methoxy-5-(4-methyl-1H-imidazol-1-yl)pyridine-2-carboxamide;
N-[(3R)-5-chloro-6-ethoxy-2,3-dihydro-1-benzofuran-3-yl]-6-methoxy-5-(4-methyl-1H-imidazol-1-yl)pyridine-2-carboxamide;
N-[(3S)-5-chloro-6-ethoxy-2,3-dihydro-1-benzofuran-3-yl]-6-methoxy-5-(4-methyl-1H-imidazol-1-yl)pyridine-2-carboxamide;
6-methoxy-5-(4-methyl-1H-imidazol-1-yl)-N-[(3R)-5-phenyl-2,3-dihydro-1-benzofuran-3-yl]pyridine-2-carboxamide;
6-methoxy-5-(4-methyl-1H-imidazol-1-yl)-N-[(3S)-5-phenyl-2,3-dihydro-1-benzofuran-3-yl]pyridine-2-carboxamide;
6-methoxy-N-[(3R)-5-methyl-2,3-dihydro-1-benzofuran-3-yl]-5-(4-methyl-1H-imidazol-1-yl)pyridine-2-carboxamide;
6-methoxy-N-[(3S)-5-methyl-2,3-dihydro-1-benzofuran-3-yl]-5-(4-methyl-1H-imidazol-1-yl)pyridine-2-carboxamide;
N-[(3R)-5,6-dimethyl-2,3-dihydro-1-benzofuran-3-yl]-6-methoxy-5-(4-methyl-1H-imidazol-1-yl)pyridine-2-carboxamide;
N-[(3S)-5,6-dimethyl-2,3-dihydro-1-benzofuran-3-yl]-6-methoxy-5-(4-methyl-1H-imidazol-1-yl)pyridine-2-carboxamide;
6-methoxy-5-(4-methyl-1H-imidazol-1-yl)-N-[(3R)-5-phenoxy-2,3-dihydro-1-benzofuran-3-yl]pyridine-2-carboxamide;
6-methoxy-5-(4-methyl-1H-imidazol-1-yl)-N-[(3S)-5-phenoxy-2,3-dihydro-1-benzofuran-3-yl]pyridine-2-carboxamide;
6-methoxy-5-(4-methyl-1H-imidazol-1-yl)-N-[(3R)-6-phenoxy-2,3-dihydro-1-benzofuran-3-yl]pyridine-2-carboxamide;
6-methoxy-5-(4-methyl-1H-imidazol-1-yl)-N-[(3S)-6-phenoxy-2,3-dihydro-1-benzofuran-3-yl]pyridine-2-carboxamide;
N-[(3S)-5-chloro-4-fluoro-2,3-dihydro-1-benzofuran-3-yl]-6-methoxy-5-(4-methyl-1H-imidazol-1-yl)pyridine-2-carboxamide;
N-[(3S)-5-chloro-6-methyl-2,3-dihydro-1-benzofuran-3-yl]-6-methoxy-5-(4-methyl-1H-imidazol-1-yl)pyridine-2-carboxamide;
2-methoxy-3-(4-methyl-1H-imidazol-1-yl)-6-({(3S)-3-[2-(trifluoromethyl)phenoxy]pyrrolidin-1-yl}carbonyl)pyridine;
N-(5-chloro-4-methyl-2,3-dihydro-1-benzofuran-3-yl)-6-methoxy-5-(4-methyl-1H-imidazol-1-yl)pyridine-2-carboxamide;
N-[(3R)-5-chloro-2,3-dihydro-1-benzofuran-3-yl]-6-methoxy-5-(4-methyl-1H-imidazol-1-yl)pyridine-2-carboxamide;
N-(5-chloro-7-methyl-2,3-dihydro-1-benzofuran-3-yl)-6-methoxy-5-(4-methyl-1H-imidazol-1-yl)pyridine-2-carboxamide;
N-[4-fluoro-5-(trifluoromethyl)-2,3-dihydro-1-benzofuran-3-yl]-6-methoxy-5-(4-methyl-1H-imidazol-1-yl)pyridine-2-carboxamide;
N-[(3R)-5-chloro-6-fluoro-2,3-dihydro-1-benzofuran-3-yl]-6-methoxy-5-(4-methyl-1H-imidazol-1-yl)pyridine-2-carboxamide;
N-(2,4-dichlorobenzyl)-6-methoxy-5-(4-methyl-1H-imidazol-1-yl)pyridine-2-carboxamide; and
6-methoxy-5-(4-methyl-1H-imidazol-1-yl)-N-[5-(trifluoromethyl)-2,3-dihydro-1-benzofuran-3-yl]pyridine-2-carboxamide,
and the pharmaceutically acceptable salts of each of the foregoing.

23. A method for the treatment of a disease or condition selected from the group consisting of neurological and psychiatric disorders comprising administering to the mammal an effective amount of compound of claim 1 or 22 or pharmaceutically acceptable salt thereof.

24. A pharmaceutical composition comprising a compound of claim 1 or 22 or a pharmaceutically acceptable salt thereof and a pharmaceutically acceptable carrier.

25. A compound according to claim 10 wherein X is N and Y is CH and a pharmaceutically acceptable salt thereof.

26. A compound according to claim 25 wherein Z is CH and a pharmaceutically acceptable salt thereof.

27. A compound according to claim 25 wherein Z is N and a pharmaceutically acceptable salt thereof.

28. A compound according to claim 10 wherein X is CH and a pharmaceutically acceptable salt thereof.

29. A compound according to claim 28 wherein Y is N and Z is CH and a pharmaceutically acceptable salt thereof.

30. A compound according to claim 28 wherein Y is CH and Z is N and a pharmaceutically acceptable salt thereof.

31. A compound according to claims 26, 27, 29 and 30 wherein R¹ is methyl, R² is hydrogen, halogen or —OR⁵ wherein R⁵ is hydrogen or C₁₋₆alkyl and a pharmaceutically acceptable salt thereof.

32. The compound according to claim 26 wherein R³ is hydrogen, C₁₋₆alkyl, or —(C(R⁶)₂)ₜ-(C₃₋₆cycloalkyl); wherein said alkyl, or cycloalkyl moiety may be optionally independently substituted with one to three fluorines; and R⁴ is C₁₋₆alkyl, —(C(R⁶)2)ₜ-(C₃₋₆cycloalkyl), —(C(R⁶)₂)ₜ-(4- to 10-membered heterocycloalkyl), —(C(R⁶)₂)ₜ-(C₆₋₁₀aryl), or —(C(R⁶)₂)ₜ-(5- to 10-membered heteroaryl); wherein said C₁-₆alkyl, or cycloalkyl, heterocycloalkyl, aryl, or heteroaryl moieties may be optionally independently substituted with one to three substituents independently selected from R⁶; or wherein R³ and R⁴ together with the nitrogen to which they are bonded form a 4- to 10-membered heterocycloalkyl, wherein said heterocycloalkyl is optionally substituted with one to three R⁶ and a pharmaceutically acceptable salt thereof.

33. The compound according to claim 32 wherein R³ is hydrogen or wherein R³ and R⁴ together with the nitrogen to which they are bonded form a 4- to 10-membered heterocycloalkyl, wherein said heterocycloalkyl is optionally substituted with one to three R⁶ and a pharmaceutically acceptable salt thereof.
